SEQUENCE LISTING (38 sequences)

SEQ ID NO: 1 - DNA, 18 bp, synthetic construct
  source 1..18: mol_type = other DNA
ataaaagcaactgttagc

SEQ ID NO: 2 - DNA, 18 bp, synthetic construct
  source 1..18: mol_type = other DNA
gtgataaaagcaactgtt

SEQ ID NO: 3 - protein, 176 aa, synthetic construct
LEPGEKPYKCPECGKSFSERSHLREHQRTHTGEKPYKCPECGKSFSTSGSLVRHQRTHTGEKPYKCPECGKSFSTHLDLIRHQRTHTGEKPYKCPECGKSFSQSGDLRRHQRTHTGEKPYKCPECGKSFSQRANLRAHQRTHTGEKPYKCPECGKSFSQKSSLIAHQRTHTGKKTS

SEQ ID NO: 4 - protein, 176 aa, synthetic construct
LEPGEKPYKCPECGKSFSTSGSLVRHQRTHTGEKPYKCPECGKSFSTHLDLIRHQRTHTGEKPYKCPECGKSFSQSGDLRRHQRTHTGEKPYKCPECGKSFSQRANLRAHQRTHTGEKPYKCPECGKSFSQKSSLIAHQRTHTGEKPYKCPECGKSFSRSDELVRHQRTHTGKKTS

SEQ ID NO: 5 - protein, 7 aa, synthetic construct
ERSHLRE

SEQ ID NO: 6 - protein, 7 aa, synthetic construct
TSGSLVR

SEQ ID NO: 7 - protein, 7 aa, synthetic construct
THLDLIR

SEQ ID NO: 8 - protein, 7 aa, synthetic construct
QSGDLRR

SEQ ID NO: 9 - protein, 7 aa, synthetic construct
QRANLRA

SEQ ID NO: 10 - protein, 7 aa, synthetic construct
QKSSLIA

SEQ ID NO: 11 - protein, 7 aa, synthetic construct
TSGSLVR

SEQ ID NO: 12 - protein, 7 aa, synthetic construct
THLDLIR

SEQ ID NO: 13 - protein, 7 aa, synthetic construct
QSGDLRR

SEQ ID NO: 14 - protein, 7 aa, synthetic construct
QRANLRA

SEQ ID NO: 15 - protein, 7 aa, synthetic construct
QKSSLIA

SEQ ID NO: 16 - protein, 7 aa, synthetic construct
RSDELVR

SEQ ID NO: 17 - DNA, 142 bp, unidentified
  source 1..142: mol_type = genomic DNA; note = Adeno-Associated Virus Type 6
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggta

SEQ ID NO: 18 - DNA, 143 bp, synthetic construct
  source 1..143: mol_type = other DNA
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggtat

SEQ ID NO: 19 - DNA, 5537 bp, synthetic construct
  source 1..5537: mol_type = other DNA
cagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgccttgcccactccctctatgcgcgctcgctcgctcggtggggcctgcggaccaaaggtccgcagacggcagagctctgctctgccggccccaccgagcgagcgagcgcgcatagagggagtgggcaaaattcacgcgtggatctgaattcaattcacgcgtggtacggtacctgtgacgtcacaggaaatgacgtaattgtccgccatcttgtaccggaagtcccgcctaccggcggcgaccggcggcatctgatttggtgtcttcttttaaattttagcgggcttttttcccgccttatgcaaatgggcagccattttaagtgttttactataattttattggttagttttgtaacggttaaaatgggcggagcgtaggcggggactacagtatatatagcacggtactgccgcagctctttctttctgggctgctttttcctggactttcttgctgttttttgtgagctaactaacaggtatttatactacttgttaacatcctaacacgcgtctcgaggaattcatgccgaaaaaaaaacgcaaagtgctagagcccggggagaaaccctataaatgtcccgagtgcggcaagtccttctcggagcggtcccacctgcgggagcatcagcgtacccacacgggcgaaaagccgtacaaatgcccagaatgcggtaaatccttcagcacctccggctccctggtgcggcatcaacggacgcatacaggagagaagccatacaaatgtcccgaatgtgggaagagttttagtacccacctggacctgatccggcaccaacgaactcacaccggtgagaagccctataagtgtccggagtgtggcaagtcgttctcgcagtccggcgacctgcggcggcaccagcgcactcatactggtgagaagccctataaatgcccggaatgtgggaagtcgtttagtcagcgggccaacctgcgggcccatcaacgtacccacaccggggaaaaaccttataagtgcccagagtgcggtaaaagcttcagccagaagtcctccctgatcgcccaccagcgcactcacaccggcaaaaaaaccagcaggcctgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttagatctaggcctgattataaagatgatgatgataaagattataaagatgatgatgataaagattataaagatgatgatgataaatgaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctggaatacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaacccccccccccccccccctgcatgcaggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 20 - DNA, 594 bp, synthetic construct
  source 1..594: mol_type = other DNA
gacaagcttcaccatcatcaccaccatcaccaccatcaccatcacgaattcctcgagctagagcccggggagaaaccctataaatgtcccgagtgcggcaagtccttctcggagcggtcccacctgcgggagcatcagcgtacccacacgggcgaaaagccgtacaaatgcccagaatgcggtaaatccttcagcacctccggctccctggtgcggcatcaacggacgcatacaggagagaagccatacaaatgtcccgaatgtgggaagagttttagtacccacctggacctgatccggcaccaacgaactcacaccggtgagaagccctataagtgtccggagtgtggcaagtcgttctcgcagtccggcgacctgcggcggcaccagcgcactcatactggtgagaagccctataaatgcccggaatgtgggaagtcgtttagtcagcgggccaacctgcgggcccatcaacgtacccacaccggggaaaaaccttataagtgcccagagtgcggtaaaagcttcagccagaagtcctccctgatcgcccaccagcgcactcacaccggcaaaaaaaccagcaggcctgtc

SEQ ID NO: 21 - DNA, 794 bp, synthetic construct
  source 1..794: mol_type = other DNA
tgccgaaaaaaaaacgcaaagtgctagagcccggggagaaaccctataagtgtccggagtgtggcaagtcgttctcgcgggccgacaacctgaccgagcatcagcgtacccacacgggcgaaaagccgtacaaatgcccagaatgcggtaaatccttcagcacctcccactccctgaccgagcatcaacggacgcatacaggagagaagccatacaaatgtcccgaatgtgggaagagttttagtcagcgggccaacctgcgggcccaccagcgcactcacaccggcgaaaaaccgtataagtgtcccgaatgcggaaagtcattttcgcagaactccaccctgaccgagcaccagcggacacataccggtgagaagccctataagtgtccggagtgtggcaagtcgttctccgaccccggccacctggtgcggcatcagcgtacccacacgggcgaaaagccgtacaaatgcccagaatgcggtaaatccttcagcaccaccggcaacctgaccgtgcatcaacggacgcatacaggagagaagccatacaaatgtcccgaatgtgggaagagttttagtacctccggcaacctggtgcggcaccagcgcactcacaccggcgaaaaaccgtataagtgtcccgaatgcggaaagtcattttcgcagctggcccacctgcgggcccaccaacgcacgcacaccggcaaaaaaaccagcgattataaagatgatgatgataaagattataaagatgatgatgataaagattataaagatgatgatgataaatga

SEQ ID NO: 22 - DNA, 384 bp, synthetic construct
  source 1..384: mol_type = other DNA
ccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttagcgggcttttttcccgccttatgcaaatgggcagccattttaagtgttttactataattttattggttagttttgtaacggttaaaatgggcggagcgtaggcggggactacagtatatatagcacggcactgccgcagctctttctttctgggctgctttttcctggactttcttgctgttttttgtgagctaactaacaggtatttatactacttgttaacatactaa

SEQ ID NO: 23 - protein, 232 aa, synthetic construct
LEPGEKPYKCPECGKSFSRADNLTEHQRTHTGEKPYKCPECGKSFSTSHSLTEHQRTHTGEKPYKCPECGKSFSQRANLRAHQRTHTGEKPYKCPECGKSFSQNSTLTEHQRTHTGEKPYKCPECGKSFSDPGHLVRHQRTHTGEKPYKCPECGKSFSTTGNLTVHQRTHTGEKPYKCPECGKSFSTSGNLVRHQRTHTGEKPYKCPECGKSFSQLAHLRAHQRTHTGKKTS

SEQ ID NO: 24 - protein, 7 aa, synthetic construct
RADNLTE

SEQ ID NO: 25 - protein, 7 aa, synthetic construct
TSHSLTE

SEQ ID NO: 26 - protein, 7 aa, synthetic construct
QRANLRA

SEQ ID NO: 27 - protein, 7 aa, synthetic construct
QNSTLTE

SEQ ID NO: 28 - protein, 7 aa, synthetic construct
DPGHLVR

SEQ ID NO: 29 - protein, 7 aa, synthetic construct
TTGNLTV

SEQ ID NO: 30 - protein, 7 aa, synthetic construct
TSGNLVR

SEQ ID NO: 31 - protein, 7 aa, synthetic construct
QLAHLRA

SEQ ID NO: 32 - DNA, 24 bp, synthetic construct
  source 1..24: mol_type = other DNA
cctgtgataaaagcaactgttagc

SEQ ID NO: 33 - DNA, 22 bp, Homo sapiens
  source 1..22: mol_type = genomic DNA
tgtgataaaagcaactgttagc

SEQ ID NO: 34 - DNA, 22 bp, Pan troglodytes
  source 1..22: mol_type = genomic DNA
tgtgataaaagcaactgttagc

SEQ ID NO: 35 - DNA, 22 bp, Pongo pygmaeus
  source 1..22: mol_type = genomic DNA
tgtgataaaagcaactgttagc

SEQ ID NO: 36 - DNA, 22 bp, Callithrix jacchus
  source 1..22: mol_type = genomic DNA
tgtgataaaagcaactgttagc

SEQ ID NO: 37 - DNA, 21 bp, Mus musculus
  source 1..21: mol_type = genomic DNA
tgtgataaagcaactgttagc

SEQ ID NO: 38 - DNA, 1422 bp, synthetic construct
  source 1..1422: mol_type = other DNA
ggccactccctctctgcgcgctcgctcgctcactgaggccgcccgggcaaagcccgggcgtcgggcgacctttggtcgcccggcctcagtgagcgagcgagcgcgcagagagggagtggccaaggtacctgtgacgtcacaggaaatgacgtaattgtccgccatcttgtaccggaagtcccgcctaccggcggcgaccggcggcatctgatttggtgtcttcttttaaattttagcgggcttttttcccgccttatgcaaatgggcagccattttaagtgttttactataattttattggttagttttgtaacggttaaaatgggcggagcgtaggcggggactacagtatatatagcacggtactgccgcagctctttctttctgggctgctttttcctggactttcttgctgttttttgtgagctaactaacaggtatttatactacttgttaacatcctaacacgcgtgaattcatgccgaaaaaaaaacgcaaagtgctagagcccggggagaaaccctataagtgtccggagtgtggcaagtcgttctcgcgggccgacaacctgaccgagcatcagcgtacccacacgggcgaaaagccgtacaaatgcccagaatgcggtaaatccttcagcacctcccactccctgaccgagcatcaacggacgcatacaggagagaagccatacaaatgtcccgaatgtgggaagagttttagtcagcgggccaacctgcgggcccaccagcgcactcacaccggcgaaaaaccgtataagtgtcccgaatgcggaaagtcattttcgcagaactccaccctgaccgagcaccagcggacacataccggtgagaagccctataagtgtccggagtgtggcaagtcgttctccgaccccggccacctggtgcggcatcagcgtacccacacgggcgaaaagccgtacaaatgcccagaatgcggtaaatccttcagcaccaccggcaacctgaccgtgcatcaacggacgcatacaggagagaagccatacaaatgtcccgaatgtgggaagagttttagtacctccggcaacctggtgcggcaccagcgcactcacaccggcgaaaaaccgtataagtgtcccgaatgcggaaagtcattttcgcagctggcccacctgcgggcccaccaacgcacgcacaccggcaaaaaaaccagcgattataaagatgatgatgataaagattataaagatgatgatgataaagattataaagatgatgatgataaatgaaggcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

(19) United States
(12) Patent Application Publication
(10) Pub. No.: US 2026/0356898 A1
(43) Pub. Date: Nov. 12, 2026

(54) NUCLEASE-FREE GENOME EDITING USING ZINC FINGER DNA BINDING DOMAINS

(71) Applicant: University of Florida Research Foundation, Incorporated, Gainesville, FL (US)

(72) Inventors: Jorg Bungert, Gainesville, FL (US); Keyun Qing, Gainesville, FL (US); Arun Srivastava, Gainesville, FL (US)

(73) Assignee: University of Florida Research Foundation, Incorporated, Gainesville, FL (US)

(21) Appl. No.: 19/113,517

(22) PCT Filed: Sep. 20, 2023
(86) PCT No.: PCT/US2023/074678

§ 371 Date: Mar. 20, 2025

Related U.S. Application Data
(60) Provisional application No. 63/376,578, filed on Sep. 21, 2022.

(51) Int. Cl. C07K 14/47; A61P 7/06; C12N 15/113; C12N 15/86

(52) U.S. Cl. CPC C07K 14/47; A61P 7/06; C12N 15/113; C12N 15/86; C12N 2310/531; C12N 2750/14143; C12N 2830/50; C12N 2830/60

(57) ABSTRACT

The disclosure relates to recombinant nucleic acids comprising polynucleotides encoding zinc-finger DNA binding domains (ZF-DBDs), and vectors comprising the same. In some embodiments, the encoded ZF-DBDs are capable of targeting a GATA motif in a Bc111A enhancer or, in other embodiments, a gamma-globin promoter. In certain embodiments, the recombinant nucleic acids further comprise a polynucleotide encoding a short hairpin RNA (shRNA), which in some embodiments is capable of targeting a Bc111A mRNA transcript. Methods of promoting globin expression, for example in the treatment of a hemoglobinopathy, comprising the administration of the recombinant nucleic acids of the disclosure and vectors comprising the recombinant nucleic acids of the disclosure to a subject are also described.

### RELATED APPLICATIONS

This application claims the benefit of the filing date of U.S. Provisional Application No. 63/376,578 filed on Sep. 21, 2022, the entire contents of which are incorporated herein by reference.

### REFERENCE TO AN ELECTRONIC SEQUENCE LISTING

The contents of the electronic sequence listing (U119770215WO00-SEQ-AXW.xml; Size: 43,706 bytes; and Date of Creation: Sep. 18, 2023) is herein incorporated by reference in its entirety.

### BACKGROUND

Human hemoglobinopathies, including β-thalassemia and sickle cell disease, are the most common single-gene inherited disorders, resulting in severe morbidity and mortality worldwide. Genome editing in hemoglobinopathies is a promising therapeutic approach for inhibiting sickling and restoring globin chain balance by promoting globin expression. However, the long-term safety of certain types of genome editing (e.g., nuclease-based genome editing), including CRISPR/Cas9-mediated approaches, remains unclear.

### SUMMARY

The present disclosure relates to compositions and methods for targeting an erythroid-specific Bcl11A enhancer and/or a gamma-globin promoter (e.g., a gamma-globin promoter of a gamma-globin gene comprising mutations characteristic of a hemoglobinopathy). In some embodiments, the compositions and methods of the disclosure result in the repression, inactivation, and/or silencing of Bcl11A enhancer activity and/or the increase of gamma-globin promoter activity, without the use of a nuclease. Such repression, inactivation, and/or silencing of Bcl11A enhancer activity and/or the increase of gamma-globin promoter activity may in some embodiments promote globin expression (e.g., by inhibiting the repression of globin expression which may result from the activity of Bcl11A) by interfering with the binding of a transcriptional regulator to the Bcl11A enhancer and/or gamma-globin promoter, respectively. In some embodiments, promoting globin expression results in a restoration of balance within the globin chain, which may in turn lead to the alleviation or amelioration of one or more signs or symptoms of a hemoglobinopathy (e.g., sickle cell disease; a thalassemia, etc.). In some embodiments, the compositions and methods of the disclosure are thus useful for treating certain diseases, for example a hemoglobinopathy (e.g., sickle cell disease; a thalassemia, etc.).

Aspects of the disclosure relate to recombinant nucleic acids comprising polynucleotides which encode zinc-finger DNA binding domains (ZF-DBDs), and vectors comprising the same. In certain embodiments, the recombinant nucleic acids and vectors of the disclosure further comprise a polynucleotide which encodes a short hairpin RNA (shRNA). Methods of promoting globin expression, for example in the treatment of a hemoglobinopathy, comprising the administration of said recombinant nucleic acids and vectors comprising the same to a subject are also described.

Aspects of the disclosure relate to a recombinant nucleic acid comprising a polynucleotide encoding a ZF-DBD. In some embodiments, the ZF-DBD encoded by the recombinant nucleic acid is capable of targeting a GATA motif in a Bcl11A enhancer. In some embodiments, the ZF-DBD encoded by the recombinant nucleic acid is capable of targeting a gamma-globin promoter.

In some embodiments, the ZF-DBD is encoded by a polynucleotide having at least 90%, at least 95%, at least 98%, or at least 99% sequence identity to the nucleic acid sequence of SEQ ID NO: 20. In some embodiments, the ZF-DBD is encoded by a polynucleotide having a nucleic acid sequence as shown in SEQ ID NO: 20. In some embodiments, the ZF-DBD is encoded by a polynucleotide having at least 90%, at least 95%, at least 98%, or at least 99% sequence identity to the nucleic acid sequence of SEQ ID NO: 21. In some embodiments, the ZF-DBD is encoded by a polynucleotide having a nucleic acid sequence as shown in SEQ ID NO: 21.

In some embodiments, a recombinant nucleic acid of the disclosure encodes a polypeptide having at least 90%, at least 95%, at least 98%, or at least 99% sequence identity to the amino acid sequence of SEQ ID NO: 3. In some embodiments, a recombinant nucleic acid of the disclosure encodes a polypeptide having the amino acid sequence as shown in SEQ ID NO: 3. In some embodiments, a recombinant nucleic acid of the disclosure encodes a polypeptide having at least 90%, at least 95%, at least 98%, or at least 99% sequence identity to the nucleic acid sequence of SEQ ID NO: 23. In some embodiments, a recombinant nucleic acid of the disclosure encodes a polypeptide having the amino acid sequence as shown in SEQ ID NO: 23.

In some embodiments, the encoded ZF-DBD comprises a polypeptide having at least 90%, at least 95%, at least 98%, or at least 99% sequence identity to the amino acid sequence of SEQ ID NO: 3. In some embodiments, the encoded ZF-DBD comprises a polypeptide having the amino acid sequence as shown in SEQ ID NO: 3. In some embodiments, the encoded ZF-DBD comprises a polypeptide having at least 90%, at least 95%, at least 98%, or at least 99% sequence identity to the amino acid sequence of SEQ ID NO: 23. In some embodiments, the encoded ZF-DBD comprises a polypeptide having the amino acid sequence as shown in SEQ ID NO: 23.

In some embodiments, a recombinant nucleic acid of the disclosure further comprises a polynucleotide encoding a shRNA. In some embodiments, the shRNA encoded by the recombinant nucleic acid is capable of targeting a Bcl11A mRNA transcript.

Aspects of the disclosure relate to a recombinant nucleic acid as described herein comprised in a vector. In some embodiments, the vector comprises a recombinant adeno-associated virus (rAAV) vector. In some embodiments, the rAAV vector is a recombinant self-complimentary AAV (scAAV) vector. In some embodiments, the rAAV vector is a recombinant AAV serotype 6 (AAV6) vector.

In some embodiments, a vector comprising a recombinant nucleic acid of the disclosure further comprises a polynucleotide encoding a regulatory element. In some embodiments, the regulatory element comprises a human parvovirus B19 promoter at map unit 6 (B19p6 promoter).

In some embodiments, a vector comprising a recombinant nucleic acid of the disclosure further comprises one or more AAV inverted terminal repeats (ITRs). In some embodiments, a vector comprising a recombinant nucleic acid of the disclosure further comprises two AAV ITRs. In some embodiments, the AAV ITR(s) are naturally-occurring AAV ITRs. In some embodiments, the AAV ITR(s) are synthetic AAV ITRs. In some embodiments, the AAV ITR(s) are from AAV serotype 6 (AAV6).

In some embodiments, a vector comprising a recombinant nucleic acid of the disclosure further comprises a polyadenylation (pA) signal. In some embodiments, the pA signal comprises a bovine growth hormone pA (BGH pA) or a human growth hormone pA (HGH pA) signal.

Aspects of the disclosure relate to a method of promoting globin expression, the method comprising administering a recombinant nucleic acid of the disclosure or a vector comprising a recombinant nucleic acid of the disclosure to a subject. In some embodiments, the subject has or is suspected of having β-thalassemia. In some embodiments, the subject has or is suspected of having sickle cell disease. In some embodiments, the subject is a human, non-human primate, non-primate mammal, or mouse subject. In some embodiments, the recombinant nucleic acid or vector is administered intramuscularly, intravenously, subcutaneously, intrathecally, intraperitoneally, or by direct injection into an organ or a tissue of the subject. In some embodiments, the globin for which expression is promoted is a gamma-globin. In some embodiments, globin expression in the subject is increased about 5-25% (e.g., 9-20%), about 10-30%, about 25-50%, or more, relative to globin expression in the subject prior to administration of the recombinant nucleic acid or the vector.

In some embodiments, a Bcl11A enhancer and/or a gamma-globin promoter is a human Bcl11A enhancer or a human gamma-globin promoter. In some embodiments, a Bcl11A enhancer and/or a gamma-globin promoter is a non-human primate Bcl11A enhancer or a non-human primate gamma-globin promoter. In some embodiments, a Bcl11A enhancer and/or a gamma-globin promoter is a non-primate mammalian Bcl11A enhancer or a non-primate mammalian gamma-globin promoter. In some embodiments, a Bcl11A enhancer and/or a gamma-globin promoter is a mouse Bcl11A enhancer or a mouse gamma-globin promoter.

Aspects of the invention relate to a host cell comprising a vector comprising a recombinant nucleic acid, as described herein. In some embodiments, a host cell is a cell (e.g., a HEK293 cell) in which rAAV vectors are manufactured (e.g., a producer cell). In some embodiments, a host cell is a cell within a subject which has been transduced by an rAAV vector of the disclosure.

Aspects of the invention relate to a vector comprising a polynucleotide having at least 90%, at least 95%, at least 98%, or at least 99% sequence identity to the nucleic acid sequence of SEQ ID NO: 19 or SEQ ID NO: 38. In some embodiments, the vector comprises a polynucleotide having a nucleic acid sequence as shown in SEQ ID NO: 19 or SEQ ID NO: 38. In some embodiments, a host cell (e.g., a producer cell) is transfected or transduced by a vector comprising the nucleic acid sequence of SEQ ID NO: 19 or SEQ ID NO: 38 (or a nucleic acid sequence having at least 90%, at least 95%, at least 98%, or at least 99% sequence identity thereto).

### BRIEF DESCRIPTION OF DRAWINGS

FIG. 1 shows the location of the erythroid-enhancer of the BCL11A gene (top panel). Within the erythroid-enhancer, three binding motifs for the GATA1 transcription factor (e.g., GATA binding motifs; e.g., BCL11A erythroid-enhancers) were identified, located +62, +58 and +55 kb from the transcription start site of BCL11A (middle panel). The sequences corresponding to the +58 kb GATA binding motif (e.g., +58 kb enhancer of BCL11A) in human, chimpanzee, orangutan, marmoset, and mouse (SEQ ID NOs: 23-27, respectively) are shown. The +58 kb GATA binding motif is conserved among humans, chimpanzees, orangutans, and marmosets, and differs by only one nucleotide (deletion of the adenine (A) in position 9) in mice.

FIGS. 2A-2B show the relative binding specificities of candidate zinc finger DNA binding domains (ZF-DBDs) to specific target sites within the +58 kb GATA binding motif. FIG. 2A shows the results for a 6ZF-DBD (amino acid sequence shown in SEQ ID NO: 3; nucleic acid sequence shown in SEQ ID NO: 20) targeting the +58 kb GATA binding motif shown in SEQ ID NO: 1. FIG. 2B shows the results for an alternative ZF-DBD (amino acid sequence shown in SEQ ID NO: 4) on an alternative target site within the +58 kb GATA binding motif (SEQ ID NO: 2). In an exemplary analysis, the alternative ZF-DBD targeting the GATA motif (top left) was predicted to bind to several different triple base pairs, which would unfavorably reduce the overall binding specificity of this ZF-DBD. Accordingly, this ZF-DBD was not pursued for further testing.

FIG. 3 shows in vitro DNA-binding affinity and specificity of a 6ZF-DBD (amino acid sequence shown in SEQ ID NO: 3; nucleic acid sequence shown in SEQ ID NO: 20) targeting the +58 kb GATA binding motif shown in SEQ ID NO: 1 (“BCL11A+58 kb ZF”), as assessed by Electrophoretic Mobility Shift Assay (EMSA).

FIG. 4 shows a schematic representation of an exemplary vector of the disclosure comprising a 6ZF-DBD targeting the +58 kb GATA binding motif in the Bcl11A enhancer.

FIGS. 5A-5B show schematic representations of exemplary vectors of the disclosure comprising a 6ZF-DBD targeting the +58 kb GATA binding motif in the Bcl11A enhancer (FIG. 5A) or 8ZF-DBD targeting the gamma-globin promoter (FIG. 5B), and a shRNA capable of targeting a Bcl11A mRNA transcript.

FIG. 6 shows human CD34+ cells transduced for 2 hours at 37 °C. A mock infection is shown at left; center shows cells transduced with AAV6-B19p6-8ZFN (2×10⁴ vgs/cell); right shows cells transduced with AAV6-B19p6-8ZFN (2×10⁵ vgs/cell).

### DETAILED DESCRIPTION

The present disclosure relates to compositions and methods for targeting an erythroid-specific Bcl11A enhancer and/or a gamma-globin promoter (e.g., a gamma-globin promoter of a gamma-globin gene comprising mutations characteristic of a hemoglobinopathy). Aspects of the disclosure relate to recombinant nucleic acids comprising a polynucleotide encoding zinc finger DNA binding domains (ZF-DBDs). In some embodiments, the encoded ZF-DBDs are capable of targeting a GATA motif in a Bcl11A enhancer or, in other embodiments, a gamma-globin promoter. Such recombinant nucleic acids may, in some embodiments, be comprised in a vector, for example a recombinant adeno-associated virus (rAAV) vector. Additional aspects of the invention relate to methods of promoting globin expression and/or treating a hemoglobinopathy (e.g., β-thalassemia, sickle cell disease) by administering a recombinant nucleic acid or vector of the disclosure to a subject.

### Zinc Finger DNA Binding Domains (ZF-DBDs)

Aspects of the disclosure relate to a recombinant nucleic acid comprising a polynucleotide encoding a zinc finger DNA binding domain (ZF-DBD). Additional aspects of the disclosure relate to a recombinant nucleic acid encoding a ZF-DBD and a short hairpin RNA (shRNA). In some embodiments, a recombinant nucleic acid of the disclosure encodes a ZF-DBD that is capable of targeting a GATA motif in a Bcl11A enhancer. In some embodiments, a recombinant nucleic acid of the disclosure encodes a ZF-DBD that is capable of targeting a gamma-globin promoter.

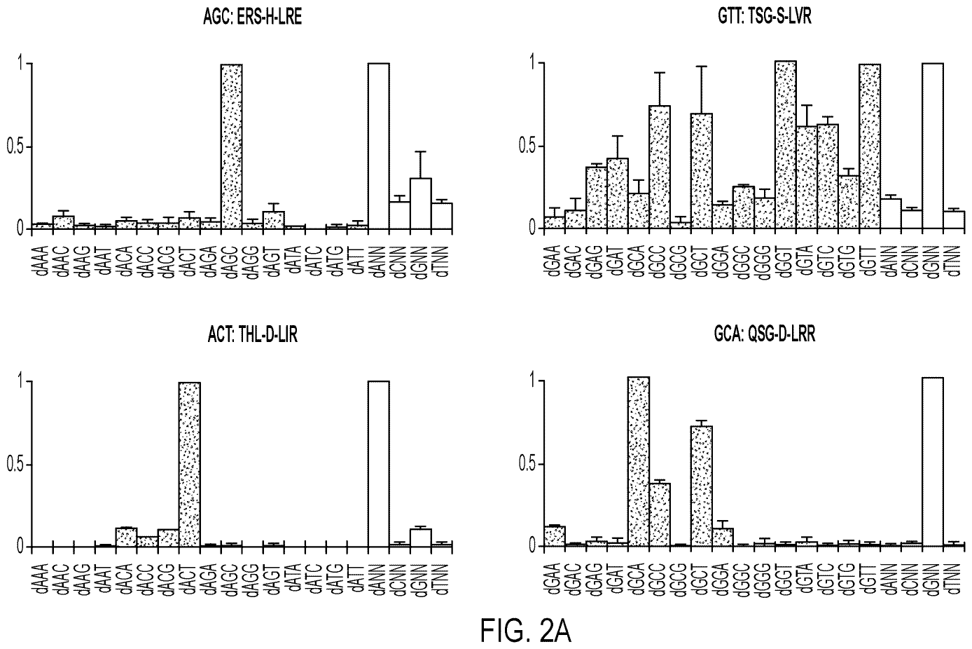

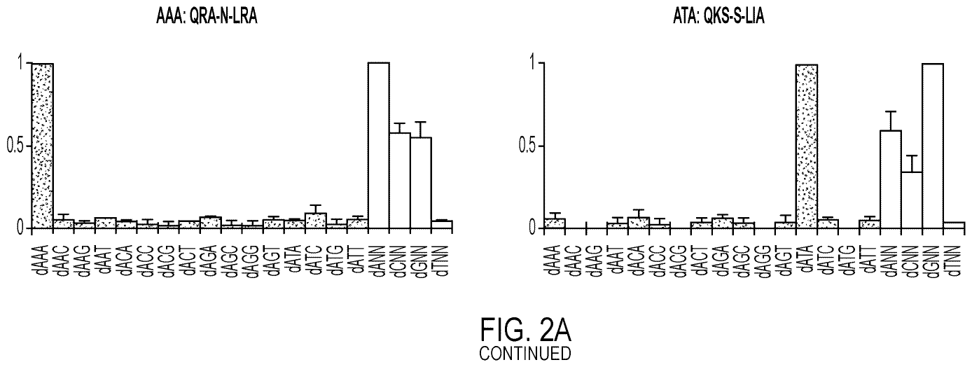

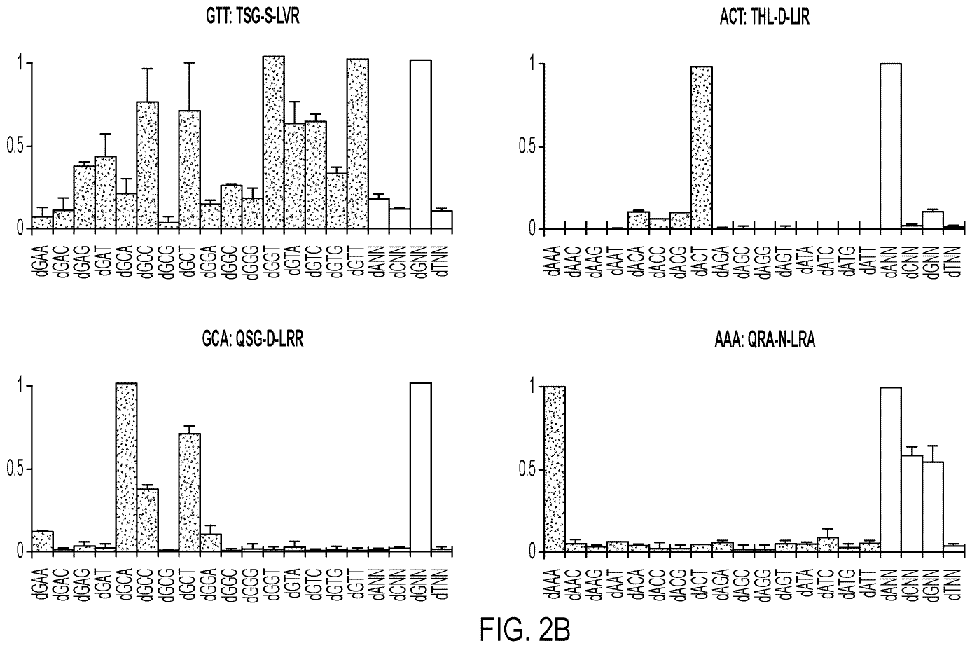

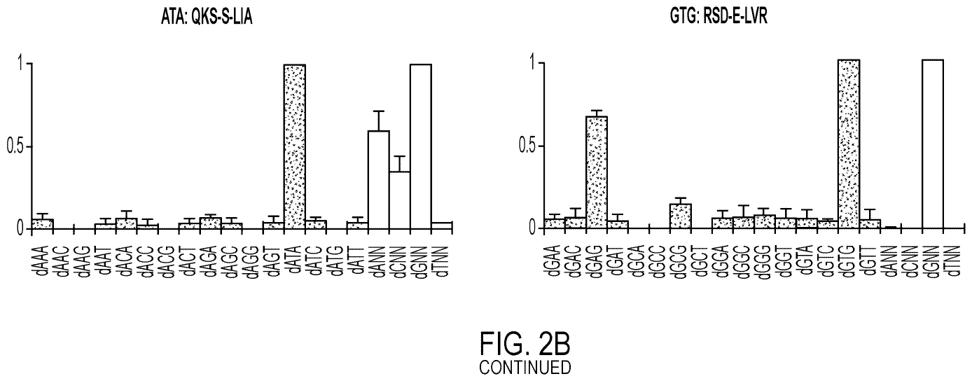

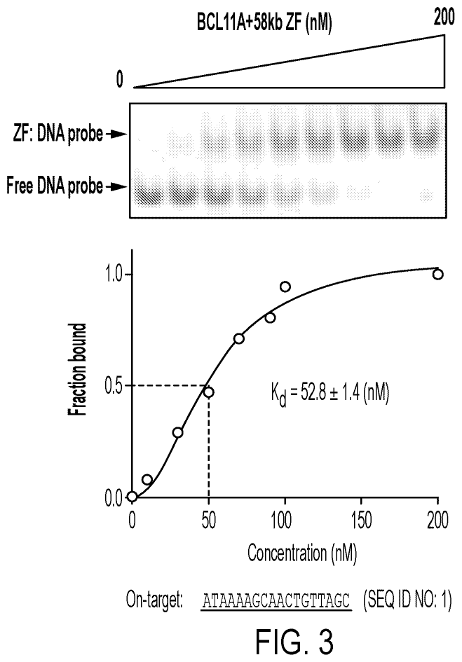

As used herein, a “ZF-DBD” refers to a zinc finger protein comprising one or more zinc finger motifs (e.g., 4-10 zinc finger motifs, for example 4, 5, 6, 7, 8, 9, or 10 zinc finger motifs) that is capable of targeting a specific sequence of DNA. By “capable of targeting”, it is meant that each zinc finger motif selectively binds to a target sequence, for example a three-base sequence of double-helical DNA. Selective binding may be measured according to various methods and assays, for example by Electrophoretic Mobility Shift Assay (EMSA) (see, e.g., Hellman & Fried, Electrophoretic mobility shift assay (EMSA) for detecting protein-nucleic acid interactions, Nat Protoc, 2:1849-861 (2007)). Such methods and assays quantify the DNA-binding properties of a protein or amino acid sequence of interest (e.g., the protein-DNA interaction), which can be reported as the fraction of target DNA molecules in a sample which are bound by the protein or amino acid sequence (e.g., from 0 to 1, or 0% to 100%, respectively; see FIGS. 2A-2B and 3). A higher fraction or percentage of target DNA molecules in a sample bound by a protein or amino acid sequence of interest indicates the selective binding of the protein or amino acid sequence to the target DNA molecules, in preference to other DNA molecules of the sample. In some embodiments, a ZF-DBD or zinc finger motif of the disclosure binds >85% of the target DNA molecules in a sample (for example 85-100%, 90-95%, 90-100%, about 85%, about 86%, about 87%, about 88%, about 89%, about 90%, about 91%, about 92%, about 93%, about 94%, about 95%, about 96%, about 97%, about 98%, about 99%, or 100%). As will be understood, the fraction or percentage of target DNA molecules in a sample bound by a protein or amino acid sequence of interest will be dependent both on the concentration of target DNA molecules and the concentration of the protein or amino acid sequence present in the sample. In some embodiments of a selective binding measurement assay, the concentration of target DNA molecules remains constant over multiple measurements of fractional binding, and the concentration of the protein or amino acid sequence of interest is progressively increased (see, e.g., FIG. 3). Such selective binding measurement assays provide the dissociation constant (K_{d}) of a particular protein or amino acid sequence of interest to a target DNA molecule. The K_{d} is the concentration of a ligand (e.g., a particular protein or amino acid sequence of interest) where half of the DNA molecules in the sample are saturated by binding with the ligand. A lower K_{d} indicates the selective binding of the protein or amino acid sequence to the target DNA molecules, in preference to other DNA molecules of the sample. In some embodiments, a ZF-DBD or zinc finger motif of the disclosure has a K_{d} for a target DNA molecule of about 50 nM (for example, 45-55 nM, 40-60 nM, about 40 nM, about 41 nM, about 42 nM, about 43 nM, about 44 nM, about 45 nM, about 46 nM, about 47 nM, about 48 nM, about 49 nM, about 50 nM, about 51 nM, about 52 nM, about 53 nM, about 54 nM, about 55 nM, about 56 nM, about 57 nM, about 58 nM, about 59 nM, or about 60 nM).

In some embodiments, a zinc finger motif comprised within a ZF-DBD of the disclosure comprises a polypeptide having the amino acid sequence of any one of SEQ ID NOs: 5-10, 11-16 or 24-31. The sequential linkage of the zinc finger motifs (e.g., SEQ ID NOs: 5-10, 11-16, or 24-31) results in an artificial zinc finger protein (e.g., a ZF-DBD) that recognizes a DNA sequence of interest, such as a GATA motif in a Bcl11A enhancer or a gamma-globin promoter. In some embodiments, a GATA motif in a Bcl11A enhancer comprises the nucleic acid sequence of SEQ ID NO: 1. In some embodiments, a GATA motif in a Bcl11A enhancer comprises the nucleic acid sequence of SEQ ID NO: 2. In some embodiments, a ZF-DBD encoded by a recombinant nucleic acid of the disclosure is capable of targeting the nucleic acid sequence of SEQ ID NO: 1. In some embodiments, a ZF-DBD encoded by a recombinant nucleic acid of the disclosure is capable of targeting the nucleic acid sequence of SEQ ID NO: 2.

In some embodiments, a ZF-DBD encoded by a recombinant nucleic acid of the disclosure is encoded by a polynucleotide having at least 90%, at least 91%, at least 92%, at least 93%, at least 94%, at least 95%, at least 96%, at least 97%, at least 98%, or at least 99% sequence identity to the nucleic acid sequence of SEQ ID NO: 20. In some embodiments, the ZF-DBD is encoded by a polynucleotide having a nucleic acid sequence as shown in SEQ ID NO: 20. In some embodiments, the ZF-DBD is encoded by a polynucleotide having no more than 60 nucleic acids which differ from (e.g., are substituted, added, or deleted relative to) the nucleic acid sequence of SEQ ID NO: 20. In some embodiments, the ZF-DBD is encoded by a polynucleotide having no more than 55 nucleic acids, no more than 50 nucleic acids, no more than 45 nucleic acids, no more than 40 nucleic acids, no more than 35 nucleic acids, no more than 30 nucleic acids, no more than 25 nucleic acids, no more than 20 nucleic acids, no more than 15 nucleic acids, no more than 10 nucleic acids, or no more than 5 nucleic acids which differ from (e.g., are substituted, added, or deleted relative to) the nucleic acid sequence of SEQ ID NO: 20. In some embodiments, the ZF-DBD is encoded by a polynucleotide having no more than 55 nucleic acids to no more than 45 nucleic acids, no more than 50 nucleic acids to no more than 40 nucleic acids, no more than 45 nucleic acids to no more than 35 nucleic acids, no more than 40 nucleic acids to no more than 30 nucleic acids, no more than 35 nucleic acids to no more than 25 nucleic acids, no more than 30 nucleic acids to no more than 20 nucleic acids, no more than 25 nucleic acids to no more than 15 nucleic acids, no more than 20 nucleic acids to no more than 10 nucleic acids, no more than 15 nucleic acids to no more than 5 nucleic acids, or no more than 5 nucleic acids to no more than 1 nucleic acid which differ from (e.g., are substituted, added, or deleted relative to) the nucleic acid sequence of SEQ ID NO: 20. In some embodiments, the ZF-DBD encoded by a polynucleotide having a nucleic acid sequence as shown in SEQ ID NO: 20 is capable of targeting a GATA motif in a Bcl11A enhancer. In some embodiments, a polynucleotide encoding a ZF-DBD of the disclosure (e.g., SEQ ID NO: 20) and a variant thereof (e.g., a polynucleotide having 90% sequence identity to the nucleic acid sequence of SEQ ID NO: 20) encode the same ZF-DBD protein, for example the polypeptide having an amino acid sequence as shown in SEQ ID NO: 3.

In some embodiments, a ZF-DBD encoded by a recombinant nucleic acid of the disclosure is encoded by a polynucleotide having at least 90%, at least 91%, at least 92%, at least 93%, at least 94%, at least 95%, at least 96%, at least 97%, at least 98%, or at least 99% sequence identity to the nucleic acid sequence of SEQ ID NO: 21. In some embodiments, the ZF-DBD is encoded by a polynucleotide having a nucleic acid sequence as shown in SEQ ID NO: 21. In some embodiments, the ZF-DBD is encoded by a polynucleotide having no more than 80 nucleic acids which differ from (e.g., are substituted, added, or deleted relative to) the nucleic acid sequence of SEQ ID NO: 21. In some embodiments, the ZF-DBD is encoded by a polynucleotide having no more than 75 nucleic acids, no more than 70 nucleic acids, no more than 65 nucleic acids, no more than 60 nucleic acids, no more than 55 nucleic acids, no more than 50 nucleic acids, no more than 45 nucleic acids, no more than 40 nucleic acids, no more than 35 nucleic acids, no more than 30 nucleic acids, no more than 25 nucleic acids, no more than 20 nucleic acids, no more than 15 nucleic acids, no more than 10 nucleic acids, or no more than 5 nucleic acids which differ from (e.g., are substituted, added, or deleted relative to) the nucleic acid sequence of SEQ ID NO: 21. In some embodiments, the ZF-DBD is encoded by a polynucleotide having no more than 80 nucleic acids to no more than 70 nucleic acids, no more than 75 nucleic acids to no more than 65 nucleic acids, no more than 70 nucleic acids to no more than 60 nucleic acids, no more than 65 nucleic acids to no more than 55 nucleic acids, no more than 60 nucleic acids to no more than 50 nucleic acids, no more than 55 nucleic acids to no more than 45 nucleic acids, no more than 50 nucleic acids to no more than 40 nucleic acids, no more than 45 nucleic acids to no more than 35 nucleic acids, no more than 40 nucleic acids to no more than 30 nucleic acids, no more than 35 nucleic acids to no more than 25 nucleic acids, no more than 30 nucleic acids to no more than 20 nucleic acids, no more than 25 nucleic acids to no more than 15 nucleic acids, no more than 20 nucleic acids to no more than 10 nucleic acids, no more than 15 nucleic acids to no more than 5 nucleic acids, or no more than 5 nucleic acids to no more than 1 nucleic acid which differ from (e.g., are substituted, added, or deleted relative to) the nucleic acid sequence of SEQ ID NO: 21. In some embodiments, the ZF-DBD encoded by a polynucleotide of SEQ ID NO: 21 is capable of targeting a gamma-globin promoter. Such ZF-DBDs which are capable of targeting a gamma-globin promoter are described, for example, in Li, et al. (2018), Fetal hemoglobin induction in sickle erythroid progenitors using a synthetic zinc finger DNA-binding domain, Haematologica, 103: e384. In some embodiments, a polynucleotide encoding a ZF-DBD of the disclosure (e.g., SEQ ID NO: 21) and a variant thereof (e.g., a polynucleotide having 90% sequence identity to the nucleic acid sequence of SEQ ID NO: 21) encode the same ZF-DBD protein, for example the polypeptide having an amino acid sequence as shown in SEQ ID NO: 23.

In some embodiments, a recombinant nucleic acid sequence of the disclosure encodes a ZF-DBD comprising an polypeptide having at least 90%, at least 91%, at least 92%, at least 93%, at least 94%, at least 95%, at least 96%, at least 97%, at least 98%, or at least 99% sequence identity to the amino acid sequence of SEQ ID NO: 3. In some embodiments, a recombinant nucleic acid sequence of the disclosure encodes a ZF-DBD comprising a polypeptide having an amino acid sequence as shown in SEQ ID NO: 3. In some embodiments, a recombinant nucleic acid sequence of the disclosure encodes a ZF-DBD comprising a polypeptide having no more than 18 amino acids which differ from (e.g., are substituted, added, or deleted relative to) the amino acid sequence of SEQ ID NO: 3. In some embodiments, a recombinant nucleic acid sequence of the disclosure encodes a ZF-DBD comprising a polypeptide having no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid(s) which differ from (e.g., are substituted, added, or deleted relative to) the amino acid sequence of SEQ ID NO: 3. In some embodiments, a recombinant nucleic acid sequence of the disclosure encodes a ZF-DBD comprising a polypeptide having no more than 18 amino acids to no more than 14 amino acids, no more than 16 amino acids to no more than 12 amino acids, no more than 14 amino acids to no more than 10 amino acids, no more than 12 amino acids to no more than 8 amino acids, no more than 10 amino acids to no more than 6 amino acids, no more than 8 amino acids to no more than 4 amino acids, no more than 6 amino acids to no more than 2 amino acids, or no more than 4 amino acids to no more than 1 amino acid which differ from (e.g., are substituted, added, or deleted relative to) the amino acid sequence of SEQ ID NO: 3.

In some embodiments, a recombinant nucleic acid sequence of the disclosure encodes a ZF-DBD comprising a polypeptide having at least 90%, at least 91%, at least 92%, at least 93%, at least 94%, at least 95%, at least 96%, at least 97%, at least 98%, or at least 99% sequence identity to the amino acid sequence of SEQ ID NO: 23. In some embodiments, a recombinant nucleic acid sequence of the disclosure encodes a ZF-DBD comprising a polypeptide having an amino acid sequence as shown in SEQ ID NO: 23. In some embodiments, a recombinant nucleic acid sequence of the disclosure encodes a ZF-DBD comprising a polypeptide having no more than 23 amino acids which differ from (e.g., are substituted, added, or deleted relative to) the amino acid sequence of SEQ ID NO: 23. In some embodiments, a recombinant nucleic acid sequence of the disclosure encodes a ZF-DBD comprising a polypeptide having no more than 23, no more than 22, no more than 21, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid(s) which differ from (e.g., are substituted, added, or deleted relative to) the amino acid sequence of SEQ ID NO: 23. In some embodiments, a recombinant nucleic acid sequence of the disclosure encodes a ZF-DBD comprising a polypeptide having no more than 24 amino acids to no more than 20 amino acids, no more than 22 amino acids to no more than 18 amino acids, no more than 20 amino acids to no more than 16 amino acids, no more than 18 amino acids to no more than 14 amino acids, no more than 16 amino acids to no more than 12 amino acids, no more than 14 amino acids to no more than 10 amino acids, no more than 12 amino acids to no more than 8 amino acids, no more than 10 amino acids to no more than 6 amino acids, no more than 8 amino acids to no more than 4 amino acids, no more than 6 amino acids to no more than 2 amino acids, or no more than 4 amino acids to no more than 1 amino acid which differ from (e.g., are substituted, added, or deleted relative to) the amino acid sequence of SEQ ID NO: 23.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, while in other embodiments, sequence identity is determined over a region of a sequence.

Identity can also refer to the degree of sequence relatedness between two sequences as determined by the number of matches between strings of two or more residues (e.g., polynucleotide or amino acid residues). Identity measures the percent of identical matches between the smaller of two or more sequences with gap alignments (if any) addressed by a particular mathematical model, algorithms, or computer program.

Identity of related nucleic acid or amino acid sequences can be readily calculated by any of the methods known to one of ordinary skill in the art. In preferred embodiments, the “percent identity” of two sequences (e.g., nucleic acid or amino acid sequences) is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. Where gaps exist between two sequences, Gapped BLAST® can be utilized, for example, as described in Altschul et al., Nucleic Acids Res. 25 (17): 3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two nucleic acid or amino acid sequences is determined by aligning the two nucleic acid or amino acid sequences, calculating the number of identical nucleic or amino acids, and dividing by the length of one of the nucleic or amino acid sequences. In some embodiments, the identity of two nucleic acid or amino acid sequences is determined by aligning the two nucleic acid or amino acid sequences and calculating the number of identical nucleic or amino acids and dividing by the length of one of the nucleic acid or amino acid sequences.

For multiple sequence alignments, computer programs including Clustal Omega (Sievers et al., Mol Syst Biol. 2011 Oct. 11; 7:539) may be used. In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539).

### Vectors Comprising Recombinant Nucleic Acids

Aspects of the disclosure relate to vectors comprising a recombinant nucleic acid of the disclosure, as described herein. As used herein, a “vector” may refer to an rAAV genome (for example, a rAAV genome comprising AAV ITRs flanking a gene of interest, and not comprising the AAV rep and cap genes) or may alternately refer to an rAAV particle (for example, comprising an rAAV genome encapsidated by viral capsid proteins). In some embodiments, the vector comprises a nucleic acid vector. In some embodiments, a vector comprising a recombinant nucleic acid of the disclosure comprises a recombinant adeno-associated virus (rAAV) or recombinant lentivirus. Additional aspects of the invention relate to a host cell comprising a vector comprising a recombinant nucleic acid, as described herein. In some embodiments, a host cell is a cell (e.g., a HEK293 cell) in which viral vectors are manufactured (e.g., a producer cell). In some embodiments, a host cell is a cell within a subject which has been transduced by a viral vector of the disclosure.

### Lentiviral Vectors

In some embodiments, a vector comprising a recombinant nucleic acid of the disclosure comprises a recombinant lentivirus genome. The lentivirus is a retrovirus, meaning it has a single stranded RNA genome with a reverse transcriptase enzyme, which functions to perform transcription of the viral genetic material upon entering the cell. Lentiviruses also have a viral envelope with protruding glycoproteins that aid in attachment to the outer membrane of a host cell. In some embodiments, the host cell comprises a lentiviral vector comprising a recombinant nucleic acid, as described herein.

Within the lentivirus genome are RNA sequences that code for specific proteins that facilitate the incorporation of the viral sequences into genome of a host cell. The “gag” gene codes for the structural components of the viral nucleocapsid proteins: the matrix (MA/p17), the capsid (CA/p24) and the nucleocapsid (NC/p7) proteins. The “pol” domain codes for the reverse transcriptase and integrase enzymes. Lastly, the “env” domain of the viral genome encodes for the glycoproteins and envelope on the surface of the virus. The ends of the genome are flanked with long terminal repeats (LTRs). LTRs are necessary for integration of the dsDNA into the host chromosome. LTRs also serve as part of the promoter for transcription of the viral genes.

In some embodiments, the env, gag, and/or pol vector(s) forming the particle do not contain a nucleic acid sequence from the lentiviral genome that expresses an envelope protein. In some embodiments, a separate vector containing a nucleic acid sequence encoding an envelope protein operably linked to a promoter is used (e.g., an env vector). In some embodiments, such env vector also does not contain a lentiviral packaging sequence. In some embodiments, the env nucleic acid sequence encodes a lentiviral envelope protein.

The native lentivirus promoter is located in the U3 region of the 3′LTR. As will be understood by those of skill in the art, the presence of the lentivirus promoter can in some embodiments interfere with heterologous promoters operably linked to a e.g. a recombinant nucleic acid of the present disclosure. To minimize such interference and better regulate the expression of recombinant nucleic acids, in some embodiments the lentiviral promoter is deleted. In some embodiments, the lentivirus vector contains a deletion within the viral promoter. After reverse transcription, such a deletion is in some embodiments transferred to the 5′ LTR, yielding a vector/provirus that is incapable of synthesizing vector transcripts from the 5′ LTR in the next round of replication.

An exemplary method for producing a recombinant lentiviral vector comprising a gene of interest (e.g., a recombinant nucleic acid of the disclosure) for administration to a subject is next described. In some embodiments, the vector components are expressed by a vector system encoding the necessary viral proteins to produce a lentivirus particle, and the vector is assembled (e.g., self-assembled) from the vector components. In some embodiments, there is at least one vector containing a nucleic acid sequence encoding the lentiviral Pol proteins necessary for reverse transcription and integration, operably linked to a promoter. In some embodiments, the Pol proteins are expressed by multiple vectors. In some embodiments, there is also a vector containing a nucleic acid sequence encoding the lentiviral Gag proteins necessary for forming a viral capsid operably linked to a promoter. In some embodiments, the gag-pol genes are on the same vector. In some embodiments, the gag nucleic acid sequence is on a separate vector than at least some of the pol nucleic acid sequence. In some embodiments, the gag nucleic acid sequence is on a separate vector from all the pol nucleic acid sequences that encode Pol proteins. In some embodiments, the lentivirus vector does not contain nucleotides from the lentiviral genome that package lentiviral RNA, referred to as the lentiviral packaging sequence.

It will be understood that selective inclusion of envelopes could result in changes in infectivity, such that the lentivirus vector could infect many different types of cells, and could be targeted to specific cell types of interest. Accordingly, in some embodiments, the envelope protein is not from the lentivirus, but from a different virus. The resultant lentivirus particle is referred to as a pseudotyped particle. In some embodiments, an env gene that encodes an envelope protein that targets an endocytic compartment such as that of the influenza virus, VSV-G, alpha viruses (Semliki forest virus, Sindbis virus), arenaviruses (lymphocytic choriomeningitis virus), flaviviruses (tick-borne encephalitis virus, Dengue virus), rhabdoviruses (vesicular stomatitis virus, rabies virus), and orthomyxoviruses (influenza virus) is used.

In some embodiments, the lentivirus is a human immunodeficiency virus (HIV1 or HIV2), a feline immunodeficiency virus (FIV), a bovine immunodeficiency virus (BIV), a caprine arthritis encephalitis virus, an equine infectious anemia virus, a jembrana disease virus, a puma lentivirus, aimian immunodeficiency virus, or a visna-maedi virus.

In some embodiments, a recombinant nucleic acid sequence encoding a ZF-DBD of the present disclosure is inserted into the empty lentiviral particles by use of a plurality of vectors (packaging vectors) each containing a nucleic acid segment of interest and a lentiviral packaging sequence necessary to package lentiviral RNA into the lentiviral particles. In some embodiments, the packaging vector contains a 5′ and 3′ lentiviral LTR with the desired nucleic acid segment inserted between them. The nucleic acid segment can be antisense molecules or, in some embodiments, encodes a ZF-DBD of the disclosure. In some embodiments, the packaging vector contains a selectable marker gene. Such marker genes are well known in the art and include green fluorescent protein (GFP), blue fluorescent protein (BFP), luciferase, LacZ, nerve growth factor receptor (NGFR), etc.

rAAV Vectors

In some embodiments, a vector comprising a recombinant nucleic acid of the disclosure comprises a rAAV genome. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ~2.3 kb- and a ~2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

rAAV vectors may comprise a nucleic acid vector (e.g., a vector comprising a recombinant nucleic acid of the disclosure). Said nucleic acid vector may comprise at a minimum (a) one or more heterologous nucleic acid regions comprising a sequence encoding a protein or polypeptide of interest (e.g., a ZF-DBD of the disclosure) and/or an RNA of interest (e.g., a shRNA) and (b) one or more regions comprising ITR sequences (e.g., wild-type ITR sequences or engineered ITR sequences) flanking the one or more heterologous nucleic acid regions. In some embodiments, the nucleic acid vector is between 4 kb and 5 kb in size (e.g., 4.2 to 4.7 kb in size). This nucleic acid vector may be encapsidated by a viral capsid, such as an AAV2 or AAV6 capsid, which may comprise a modified capsid protein as described herein.

In some embodiments, an rAAV vector comprises AAV6 capsid protein(s). In some embodiments, an rAAV vector comprises modified AAV6 capsid protein(s). In some embodiments, modified AAV6 capsid protein(s) comprise one or more amino acid mutations (e.g., amino acid substitutions, additions, or deletions). In some embodiments, the modified AAV6 capsid protein(s) comprise the following amino acid substitutions: Y444F; Y733P; and T492V. In some embodiments, the modified AAV6 capsid protein(s) comprise the following amino acid substitutions: Y705F; Y731F; and T492V.

In some embodiments, the rAAV vector comprising modified AAV6 capsid protein(s) (e.g., Y444F+Y733F+T492V or Y705F+Y731F+T492V) is highly efficient in transducing primary human hematopoietic stem cells. In some embodiments, the rAAV vector comprising modified AAV6 capsid protein(s) (e.g., Y444F+Y733F+T492V or Y705F+Y731F+T492V) is more efficient in transducing primary human hematopoietic stem cells than an rAAV vector comprising unmodified AAV6 capsid protein(s). In some embodiments, the rAAV vector comprising modified AAV6 capsid protein(s) (e.g., Y444F+Y733F+T492V or Y705F+Y731F+T492V) is more efficient in transducing primary human hematopoietic stem cells than an rAAV vector comprising modified or unmodified AAV capsid protein(s) of a serotype other than AAV6.

In some embodiments, the rAAV vector comprising modified AAV6 capsid protein(s) (e.g., Y444F+Y733F+T492V or Y705F+Y731F+T492V) is safe for nuclease-free genome editing (e.g., for human hemoglobinopathies) (e.g., does not result in undesirable or harmful effects upon administration to a subject). In some embodiments, the rAAV vector comprising modified AAV6 capsid protein(s) (e.g., Y444F+Y733F+T492V or Y705F+Y731F+T492V) is safer for nuclease-free genome editing (e.g., for human hemoglobinopathies) than an rAAV vector comprising unmodified AAV6 capsid protein(s). In some embodiments, the rAAV vector comprising modified AAV6 capsid protein(s) (e.g., Y444F+Y733F+T492V or Y705F+Y731F+T492V) is safer for nuclease-free genome editing (e.g., for human hemoglobinopathies) than an rAAV vector comprising modified or unmodified AAV capsid protein(s) of a serotype other than AAV6.

In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector. In some embodiments, the rAAV vector is a recombinant self-complimentary AAV (scAAV) vector.

Accordingly, in some embodiments, an rAAV vector comprises a viral capsid and a nucleic acid vector as described herein, which is encapsidated by the viral capsid. In some embodiments, the nucleic acid vector comprises (1) one or more heterologous nucleic acid regions comprising a sequence encoding a protein or polypeptide of interest (e.g., a ZF-DBD of the disclosure) and/or an RNA of interest (e.g., a shRNA), (2) one or more nucleic acid regions comprising a sequence that facilitates expression of the heterologous nucleic acid region (e.g., a B19p6 promoter), and (3) one or more ITR sequences. In some embodiments, the nucleic acid vector comprises one or more heterologous nucleic acid regions comprising a sequence encoding a protein, polypeptide, or RNA of interest (e.g., a ZF-DBD or shRNA of the disclosure) operably linked to a promoter, wherein the one or more heterologous nucleic acid regions are flanked on each side with an ITR sequence.

In some embodiments, a rAAV vector comprising a recombinant nucleic acid of the disclosure further comprises one or more AAV ITRs. In some embodiments, a rAAV vector comprising a recombinant nucleic acid of the disclosure further comprises two AAV ITRs. In some embodiments, the AAV ITR(s) are naturally-occurring AAV ITRs. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV6. In some embodiments, the AAV ITR(s) are from AAV6. In some embodiments, a vector comprising a recombinant nucleic acid of the disclosure further comprises an AAV ITR having the nucleic acid sequence of SEQ ID NO: 17.

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93 (24): 14082-7; and Curtis A. Machida. Methods in Molecular Medicine™. Viral Vectors for Gene Therapy Methods and Protocols. 10.1385/1-59259-304-6:201 @ Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, a vector comprising a recombinant nucleic acid of the disclosure further comprises one or more synthetic AAV ITRs. As used herein, a “synthetic” AAV ITR is one which contains one or more substitutions, additions, or mutations relative to a wild-type AAV ITR of the same serotype. In some embodiments, the synthetic AAV ITR is a synthetic AAV6 ITR. In some embodiments, the synthetic AAV6 ITR contains an additional thymine (T) nucleotide at its 3′ end, relative to a wild-type AAV6 ITR. In some embodiments, a vector comprising a recombinant nucleic acid of the disclosure further comprises an AAV ITR having the nucleic acid sequence of SEQ ID NO: 18. In some embodiments, a self-complementary recombinant AAV6 vector, as described herein, comprises one wild-type AAV6 ITR and one synthetic AAV6 ITR. In some embodiments, a self-complementary recombinant AAV6 vector, as described herein, comprises one wild-type AAV6 ITR having the nucleic acid sequence of SEQ ID NO: 17 and one synthetic AAV6 ITR having the nucleic acid sequence of SEQ ID NO: 18.

In some embodiments, the nucleic acid vector comprises a pTR-UF-11 plasmid backbone, which is a plasmid that contains AAV2 ITRs. This plasmid is commercially available from the American Type Culture Collection (ATCC MBA-331).

Methods of producing rAAV particles are known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the nucleic acid vector sequence may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more belper plasmids includes a first helper plasmid comprising a rep gene and a cap gene and a second helper plasmid comprising a E1a gene, a E1b gene, a E4 gene, a E2a gene, and a VA gene. In some embodiments, the rep gene is a rep gene derived from AAV2 and the cap gene is a cap gene derived from AAV2 and includes modifications to the gene in order to produce a modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG (R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and B4 genes under the transcriptional control of their native promoters. The HEK293, BHK, or Sf9 cells are then incubated for at least 60 hours to allow for rAAV particle production.

In some embodiments, a host cell (e.g., a HEK293, BHK, or Sf9 cell) comprises an AAV vector comprising a recombinant nucleic acid, as described herein.

### Regulatory Elements

In some embodiments, a vector comprising a recombinant nucleic acid as described herein may comprise a polynucleotide encoding one or more regulatory elements. A polynucleotide encoding a regulatory element refers to a nucleotide sequence or structural component of a nucleic acid vector which is involved in the regulation of expression of components of the nucleic acid vector (e.g., a polynucleotide encoding a ZF-DBD). Regulatory elements include, but are not limited to, promoters, enhancers, silencers, insulators, response elements, initiation sites, termination signals, ribosome binding sites, and polyadenylation elements. In some embodiments, a vector comprising a recombinant nucleic acid as described herein further comprises a polynucleotide encoding a promoter. In some embodiments, a vector comprising a recombinant nucleic acid as described herein further comprises a polyadenylation (pA) signal. In some embodiments, the pA signal comprises a bovine growth hormone pA (BGH pA) or a human growth hormone pA (HGH pA) signal.

Promoters include constitutive promoters, inducible promoters, tissue-specific promoters, cell type-specific promoters, and synthetic promoters. For example, a vector comprising a recombinant nucleic acid as described herein may include polynucleotides encoding viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter.

Inducible promoters or other inducible regulatory elements may also be used to achieve desired expression levels of a therapeutic molecule (e.g., a protein or polypeptide of interest). Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue- and cell-specific promoters or other tissue- or cell-specific regulatory elements are also contemplated herein. Synthetic promoters are also contemplated herein. A synthetic promoter may comprise, for example, regions of known promoters, regulatory elements, transcription factor binding sites, enhancer elements, repressor elements, and the like.

In some embodiments, a vector comprising a recombinant nucleic acid of the disclosure comprises a nucleic acid encoding a human parvovirus B19 promoter at map unit 6 (B19p6 promoter). The B19p6 promoter has been shown to be implicated in autonomous replication competence and erythroid specificity to AAV in primary human hematopoietic progenitor cells (see, e.g., Wang, et al. (1995), Parvovirus B19 promoter at map unit 6 confers autonomous replication competence and erythroid specificity to adeno-associated virus 2 in primary human hematopoietic progenitor cells, PNAS, 92 (26): 12416-420). In some embodiments, a B19p6 promoter comprised in a vector of the disclosure comprises the nucleic acid sequence of SEQ ID NO: 22.

In some embodiments, a vector comprising a recombinant nucleic acid as described herein may comprise a hybrid AAV6-B19p6 rAAV vector, wherein the native AAV6 promoter is replaced by the B19p6 promoter. AAV6-B19p6 rAAV vectors are described in detail in International Publication Number WO 2016/134338 A1, incorporated herein by reference in its entirety.

Aspects of the invention relate to a rAAV vector comprising a polynucleotide having at least 90%, at least 91%, at least 92%, at least 93%, at least 94%, at least 95%, at least 96%, at least 97%, at least 98%, or at least 99% sequence identity to the nucleic acid sequence of SEQ ID NO: 19 or SEQ ID NO: 38. In some embodiments, the rAAV vector comprises a polynucleotide having a nucleic acid sequence as shown in SEQ ID NO: 19 or SEQ ID NO: 38. In some embodiments, the rAAV vector comprises a polynucleotide having no more than 554 nucleic acids which differ from (e.g., are substituted, added, or deleted relative to) the nucleic acid sequence of SEQ ID NO: 19 or SEQ ID NO: 38. In some embodiments, the rAAV vector comprises a polynucleotide having no more than 550 nucleic acids, 525 nucleic acids, 500 nucleic acids, 475 nucleic acids, 450 nucleic acids, 425 nucleic acids, 400 nucleic acids, 375 nucleic acids, 350 nucleic acids, 325 nucleic acids, 300 nucleic acids, 275 nucleic acids, 250 nucleic acids, 225 nucleic acids, 200 nucleic acids, 175 nucleic acids, 150 nucleic acids, 125 nucleic acids, 100 nucleic acids, 75 nucleic acids, 50 nucleic acids, or 25 nucleic acids which differ from (e.g., are substituted, added, or deleted relative to) the nucleic acid sequence of SEQ ID NO: 19 or SEQ ID NO: 38. In some embodiments, the rAAV vector comprises a polynucleotide having no more than 554 nucleic acids to no more than 500 nucleic acids, no more than 525 nucleic acids to no more than 475 nucleic acids, no more than 500 nucleic acids to no more than 450 nucleic acids, no more than 475 nucleic acids to no more than 425 nucleic acids, no more than 450 nucleic acids to no more than 400 nucleic acids, no more than 425 nucleic acids to no more than 375 nucleic acids, no more than 400 nucleic acids to no more than 350 nucleic acids, no more than 375 nucleic acids to no more than 325 nucleic acids, no more than 350 nucleic acids to no more than 300 nucleic acids, no more than 325 nucleic acids to no more than 275 nucleic acids, no more than 300 nucleic acids to no more than 250 nucleic acids, no more than 275 nucleic acids to no more than 225 nucleic acids, no more than 250 nucleic acids to no more than 200 nucleic acids, no more than 225 nucleic acids to no more than 175 nucleic acids, no more than 200 nucleic acids to no more than 150 nucleic acids, no more than 175 nucleic acids to no more than 125 nucleic acids, no more than 150 nucleic acids to no more than 100 nucleic acids, no more than 125 nucleic acids to no more than 75 nucleic acids, no more than 100 nucleic acids to no more than 50 nucleic acids, no more than 75 nucleic acids to no more than 25 nucleic acids, or no more than 50 nucleic acids to no more than 1 nucleic acid which differ from (e.g., are substituted, added, or deleted relative to) the nucleic acid sequence of SEQ ID NO: 19 or SEQ ID NO: 38. In some embodiments, a host cell (e.g., a producer cell) is transfected or transduced by a vector comprising the nucleic acid sequence of SEQ ID NO: 19 or SEQ ID NO: 38 (or a nucleic acid sequence having at least 90%, at least 95%, at least 98%, or at least 99% sequence identity thereto).

### Therapeutic and Detectable Molecules

In some embodiments, a vector comprising a recombinant nucleic acid of the disclosure further comprises a nucleic acid encoding an additional therapeutic molecule (e.g., in addition to a ZF-DBD or shRNA of the disclosure). In some embodiments, the additional therapeutic molecule comprises a therapeutic or diagnostic protein or polypeptide. In some embodiments, a therapeutic or diagnostic protein or polypeptide is an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic agent, an enzyme, a bone morphogenetic protein, a nuclease, guide RNA or other nucleic acid or protein used for gene editing, an Fc-fusion protein, an anticoagulant, or a protein or polypeptide that can be detected using a laboratory test.

In some embodiments, a vector comprising a recombinant nucleic acid of the disclosure further comprises an additional polynucleotide which encodes a detectable molecule. A detectable molecule is a molecule that can be visualized (e.g., using a naked eye, under a microscope, or using a light detection device such as a camera). In some embodiments, the detectable molecule is a fluorescent molecule, a bioluminescent molecule, or a molecule that provides color (e.g., β-galactosidase, β-lactamase, β-glucuronidase, or spheroidenone). In some embodiments, the detectable molecule is a fluorescent, bioluminescent or enzymatic protein or functional peptide or polypeptide thereof.

In some embodiments, a fluorescent protein is a blue fluorescent protein, a cyan fluorescent protein, a green fluorescent protein, a yellow fluorescent protein, an orange fluorescent protein, a red fluorescent protein, or a functional peptide or polypeptide thereof. A blue fluorescent protein may be azurite, EBFP, EBFP2, mTagBFP, or Y66H. A cyan fluorescent protein may be ECFP, AmCyan1, Cerulean, CyPet, mECFP, Midori-ishi Cyan, mTFP1, or TagCFP. A Green fluorescent protein may be AcGFP, Azami Green, EGFP, Emarald, GFP or a mutated form of GFP (e.g., GFP-S65T, mWasabi, Stemmer, Superfolder GFP, TagGFP, TurboGFP, or ZsGreen). A yellow fluorescent protein may be EYFP, mBanana, mCitrine, PhiYFp, TagYFP, Topaz, Venus, YPet, or ZsYellow1. An orange fluorescent protein may be DsRed, RFP, DsRed2, DsRed-Express, Ds-Red-monomer, Tomato, tdTomato, Kusabira Orange, mKO2, mOrange, mOrange2, mTangerine, TagRFP, or TagRFP-T. A red fluorescent protein may be AQ142, AsRed2, dKeima-Tandem, HcRed1, tHcRed, Jred, mApple, mCherry, mPlum, mRaspberry, mRFP1, mRuby or mStrawberry.

In some embodiments, a detectable molecule is a bioluminescent protein or a functional peptide or polypeptide thereof. Non-limiting examples of bioluminescent proteins are firefly luciferase, click-beetle luciferase, Renilla luciferase, and luciferase from Oplophorus gracilirostris.

In some embodiments, a detectable molecule may be any polypeptide or protein that can be detected using methods known in the art. Non-limiting methods of detection are fluorescence imaging, luminescent imaging, bright field imaging, and imaging facilitated by immunofluorescence or immunohistochemical staining.

### Short Hairpin RNAs

In some embodiments, a vector comprising a recombinant nucleic acid of the disclosure further comprises a polynucleotide encoding a short hairpin RNA (shRNA). shRNAs are known in the art. Additional aspects of the disclosure relate to a recombinant nucleic acid encoding a zinc finger DNA binding domain (ZF-DBD) and a short hairpin RNA (shRNA), wherein the ZF-DBD is capable of targeting a γ-globin promoter and the shRNA is capable of targeting a Bcl11A mRNA transcript. Briefly, a shRNA is an artificial RNA molecule with a tight hairpin turn that can be used to silence target gene expression via RNA interference (RNAi). In some embodiments, a vector comprising a recombinant nucleic acid of the disclosure further comprises a polynucleotide encoding a shRNA that is capable of targeting a Bcl11A mRNA transcript. In some embodiments, the encoded shRNA which targets a Bcl11A mRNA transcript reduces (e.g., lessens), inactivates, and/or silences (e.g., eliminates) expression of the Bcl11A protein, relative to the expression of the Bcl11A protein in the absence of the shRNA. In some embodiments, Bcl11A protein expression is reduced by 1-5%, 3-10%, 8-20%, 15-40%, 20-60%, 30-50%, 40-75%, 55-80%, 70-90%, 75-95%, or 80-100%, relative to the expression of the Bcl11A protein in the absence of the shRNA.

### Methods of Promoting Globin Expression

Aspects of the disclosure relate to methods of promoting globin expression by administering a recombinant nucleic acid as described herein or a vector comprising a recombinant nucleic acid as described herein to a subject. In some embodiments, the globin is gamma-globin.

In some embodiments, the subject is a human, non-human primate, non-primate mammal, or mouse subject. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. In some embodiments, the subject is a human child (e.g., a human being less than 18 years of age). In some embodiments, the subject is a mouse subject. Non-limiting examples of non-primate mammalian subjects include domesticated animals such as dogs and cats; livestock such as horses, cattle, pigs, sheep, goats, and chickens; and other animals such as mice, rats, guinea pigs, and hamsters.

In some embodiments, “administering” or “administration” means providing a material to a subject in a manner that is pharmacologically useful. In some embodiments, a recombinant nucleic acid as described herein or a vector comprising a recombinant nucleic acid as described herein is administered to a subject enterally. In some embodiments, an enteral administration of the recombinant nucleic acid as described herein or the vector comprising a recombinant nucleic acid as described herein is oral. In some embodiments, a recombinant nucleic acid as described herein or a vector comprising a recombinant nucleic acid as described herein is administered to the subject parenterally. In some embodiments, a recombinant nucleic acid as described herein or a vector comprising a recombinant nucleic acid as described herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, a recombinant nucleic acid as described herein or a vector comprising a recombinant nucleic acid as described herein is administered to the subject by injection into the hepatic artery or portal vein. In some embodiments, a recombinant nucleic acid as described herein or a vector comprising a recombinant nucleic acid as described herein is administered intramuscularly, intravenously, subcutaneously, intrathecally, intraperitoneally, or by direct injection into an organ or a tissue of the subject.

Any recombinant nucleic acid or vector comprising a recombinant nucleic acid as disclosed herein may be comprised within a pharmaceutical composition comprising a pharmaceutically-acceptable carrier or may be comprised within a pharmaceutically-acceptable carrier. The term “carrier” refers to a diluent, adjuvant, excipient, or vehicle with which the recombinant nucleic acid as described herein or the vector comprising a recombinant nucleic acid as described herein is comprised or administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases), and solutions or compositions thereof. Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of compositions comprising recombinant nucleic acids or vectors comprising recombinant nucleic acids to human subjects.

Typically, such compositions may contain at least about 0.1% of the therapeutic agent (e.g., recombinant nucleic acid or vector comprising the same) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., recombinant nucleic acid or vector comprising the same) in each therapeutically-useful composition may be prepared in such a way that a suitable dosage will be obtained in any given unit dose of the compound. Factors such as solubility, bioavailability, biological half-life, route of administration, and product shelf life, as well as other pharmacological considerations, will be contemplated by one skilled in the art of preparing such pharmaceutical formulations, and as such, a variety of dosages and treatment regimens may be designed.

In some embodiments, a recombinant nucleic acid as described herein or a vector comprising a recombinant nucleic acid as described herein is administered to a subject to promote globin expression. In some embodiments, the globin is gamma (γ)-globin. The gamma-globin genes (HBG1 and HBG2) are normally expressed in the fetal liver, spleen and bone marrow. The two types of gamma chains differ at residue 136 where glycine is found in the G-gamma product (HBG2) and alanine is found in the A-gamma product (HBG1). The two gamma chains together with two alpha chains constitute fetal hemoglobin (HbF) which is normally replaced by adult hemoglobin (HbA) in the year following birth. However, in the non-pathological condition known as hereditary persistence of fetal hemoglobin (HPFH), gamma globin expression is continued into adulthood. Additionally, in cases of beta-thalassemia and related conditions gamma chain production may be maintained, possibly as a mechanism to compensate for the mutated beta-globin which characterizes these conditions. In some embodiments, a recombinant nucleic acid as described herein or a vector comprising a recombinant nucleic acid as described herein which is administered to a subject promotes gamma-globin expression in the subject. In some embodiments, such administration which promotes gamma-globin expression in the subject treats a disease or disorder in the subject.

In some embodiments, a recombinant nucleic acid as described herein or a vector comprising a recombinant nucleic acid as described herein is administered to a subject to treat a disease or disorder. To “treat” a disease or disorder, as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of a vector may be an amount of the vector that is capable of transferring an expression construct (e.g., a recombinant nucleic acid) to a host organ, tissue, or cell. A therapeutically acceptable amount may be an amount that is capable of treating a disease, e.g., a beta-thalassemia or sickle cell disease. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, the concentration of vectors (e.g., viral particles) comprising a recombinant nucleic acid of the disclosure administered to a subject (either directly or in a composition) may be on the order ranging from 10⁶ to 10¹¹ particles/ml or 10³ to 10¹¹ particles/ml, or any values therebetween for either range, such as for example, about 10⁶, 10⁷, 10⁸, 10⁹, 10¹⁰, or 10¹¹ particles/ml. In some embodiments, vectors of a higher concentration than 10¹¹ particles/ml are administered. The vectors or compositions can be administered as a single dose, or divided into two or more administrations as may be required to achieve therapy of the particular disease or disorder being treated. In some embodiments, 0.0001 ml to 10 ml are delivered to a subject. In some embodiments, the number of vectors administered to a subject may be on the order ranging from 10⁶-10¹¹ vgs/kg body mass of the subject, or any values therebetween (e.g., 10⁶, 10⁷, 10⁸, 10⁹, 10¹⁰, or 10¹¹ vgs/kg). In some embodiments, the volume of a composition comprising a vector and/or recombinant nucleic acid of the disclosure delivered to a subject (e.g., via one or more routes of administration as described herein) is 0.0001 ml to 10 ml.

In some embodiments, a composition disclosed herein (e.g., comprising a recombinant nucleic acid or vector comprising the same) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject.

In some embodiments, the subject has or is suspected of having a hemoglobinopathy. A hemoglobinopathy is a disease characterized by one or more mutation(s) in the genome that results in abnormal structure of one or more of the globin chains of the hemoglobin molecule. Exemplary hemoglobinopathies include hemolytic anemia, sickle cell disease, and thalassemia.

In some embodiments, the subject has or is suspected of having sickle cell disease. Sickle cell disease is characterized by the presence of abnormal, sickle-shaped hemoglobins, which can result in severe infections, severe pain, stroke, and an increased risk of death. Subjects having sickle cell disease can be identified, e.g., using one or more of: a complete blood count, a blood film, hemoglobin electrophoresis, and genetic testing. In some embodiments, administration of a recombinant nucleic acid or vector comprising a recombinant nucleic acid as described herein to a subject having sickle cell disease results in reactivation of the fetal gamma-globin gene. In some embodiments, administration of a recombinant nucleic acid or vector comprising a recombinant nucleic acid as described herein to a subject having sickle cell disease results in an increase in globin (e.g., gamma-globin) expression (e.g., via reactivation of the fetal gamma-globin gene). In some embodiments, reactivation of the fetal gamma-globin gene and/or an increase in globin expression in a subject having sickle cell disease results in the alleviation or amelioration of one or more signs or symptoms associated with sickle cell disease. Such signs or symptoms may include, but are not limited to, cell sickling, dyserythropoiesis, anemia, splenomegaly, marrow expansion, bone deformities, and accumulation of iron. Physical symptoms of sickle cell disease may additionally include fatigue, episodic pain (pain crises), swelling of hands and feet, frequent infections, delayed growth or puberty, and/or vision problems. In some embodiments, following administration of a recombinant nucleic acid or vector comprising a recombinant nucleic acid as described herein to a subject having sickle cell disease, one or more signs or symptoms of sickle cell disease is alleviated and/or ameliorated (e.g., a subject may have reduced pain crises per year, relative to the number of pain crises per year experienced prior to administration of a recombinant nucleic acid or vector comprising a recombinant nucleic acid as described herein). In some embodiments, following administration of a recombinant nucleic acid or vector comprising a recombinant nucleic acid as described herein to a subject having sickle cell disease, globin (e.g., gamma-globin) expression in the subject is increased about 9-20%, relative to globin expression prior to administration of the recombinant nucleic acid or the vector. In some embodiments, following administration of a recombinant nucleic acid or vector comprising a recombinant nucleic acid as described herein to a subject having sickle cell disease, globin (e.g., gamma-globin) expression in the subject is increased about 5-25%, relative to globin expression prior to administration of the recombinant nucleic acid or the vector. In some embodiments, following administration of a recombinant nucleic acid or vector comprising a recombinant nucleic acid as described herein to a subject having sickle cell disease, globin (e.g., gamma-globin) expression in the subject is increased about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%, relative to globin expression prior to administration of the recombinant nucleic acid or the vector. In some embodiments, following administration of a recombinant nucleic acid or vector comprising a recombinant nucleic acid as described herein to a subject having sickle cell disease, globin (e.g., gamma-globin) expression in the subject is increased about 9%, relative to globin expression prior to administration of the recombinant nucleic acid or the vector. In some embodiments, following administration of a recombinant nucleic acid or vector comprising a recombinant nucleic acid as described herein to a subject having sickle cell disease, globin (e.g., gamma-globin) expression in the subject is increased about 5-8%, about 7-10%, about 9-12%, about 11-14%, about 13-16%, about 15-18%, about 17-20%, about 19-22%, or about 21-25%, relative to globin expression prior to administration of the recombinant nucleic acid or the vector. In some embodiments, following administration of a recombinant nucleic acid or vector comprising a recombinant nucleic acid as described herein to a subject having sickle cell disease, globin (e.g., gamma-globin) expression in the subject is increased more than 25%, relative to globin expression prior to administration of the recombinant nucleic acid or the vector. In some embodiments, wherein fetal hemoglobin (e.g., gamma-globin) levels are about 20% (e.g., about 15-25%) of the total globin level in a subject (e.g., following administration of a recombinant nucleic acid or vector comprising a recombinant nucleic acid as described herein), sickling is inhibited in the subject.

In some embodiments, the subject has or is suspected of having a thalassemia. Thalassemias are a group of autosomal recessive diseases characterized by a reduction in the amount of hemoglobin produced. Symptoms include iron overload, infection, bone deformities, enlarged spleen, and cardiac disease. The subgroups of thalassemias include alpha-thalassemia, beta-thalassemia, and delta thalassemia. Subjects having a thalassemia may be identified, e.g., using one or more of: complete blood count, hemoglobin electrophoresis, Fe Binding Capacity, urine urobilin and urobilogen, peripheral blood smear, hematocrit, and genetic testing. In some embodiments, the subject has or is suspected of having β-thalassemia. In some embodiments, administration of a recombinant nucleic acid or vector comprising a recombinant nucleic acid as described herein to a subject having a thalassemia (e.g., β-thalassemia) results in reactivation of the fetal gamma-globin gene. In some embodiments, administration of a recombinant nucleic acid or vector comprising a recombinant nucleic acid as described herein to a subject having a thalassemia results in an increase in globin (e.g., gamma-globin) expression (e.g., via reactivation of the fetal gamma-globin gene). In some embodiments, administration of a recombinant nucleic acid or vector comprising a recombinant nucleic acid as described herein to a subject having a thalassemia results in a balancing of the globin chain (e.g., via reactivation of the fetal gamma-globin gene, and/or an increase in globin expression). In some embodiments, a balancing of the globin chain comprises a reduction in the ratio between alpha-globin and beta-globin. In some embodiments, reactivation of the fetal gamma-globin gene and/or an increase in globin expression and/or a balancing of the globin chain in a subject having a thalassemia results in the alleviation or amelioration of one or more signs or symptoms associated with thalassemias. Such signs or symptoms may include, but are not limited to, cell sickling, dyserythropoiesis, anemia, splenomegaly, marrow expansion, bone deformities, and accumulation of iron. Physical symptoms of a thalassemia (e.g., β-thalassemia) may additionally include fatigue, weakness, pale or yellowish skin, facial bone deformities, slow growth, abdominal swelling, and/or dark urine. In some embodiments, following administration of a recombinant nucleic acid or vector comprising a recombinant nucleic acid as described herein to a subject having a thalassemia (e.g., β-thalassemia), one or more signs or symptoms of the thalassemia (e.g., β-thalassemia) is alleviated and/or ameliorated (e.g., a subject may have reduced fatigue, relative to the level of fatigue experienced prior to administration of a recombinant nucleic acid or vector comprising a recombinant nucleic acid as described herein). In some embodiments, following administration of a recombinant nucleic acid or vector comprising a recombinant nucleic acid as described herein to a subject having a thalassemia, globin (e.g., gamma-globin) expression in the subject is increased about 9-20%, relative to globin expression prior to administration of the recombinant nucleic acid or the vector. In some embodiments, following administration of a recombinant nucleic acid or vector comprising a recombinant nucleic acid as described herein to a subject having a thalassemia, globin (e.g., gamma-globin) expression in the subject is increased about 5-25%, relative to globin expression prior to administration of the recombinant nucleic acid or the vector. In some embodiments, following administration of a recombinant nucleic acid or vector comprising a recombinant nucleic acid as described herein to a subject having a thalassemia, globin (e.g., gamma-globin) expression in the subject is increased about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%, relative to globin expression prior to administration of the recombinant nucleic acid or the vector. In some embodiments, following administration of a recombinant nucleic acid or vector comprising a recombinant nucleic acid as described herein to a subject having a thalassemia, globin (e.g., gamma-globin) expression in the subject is increased about 9%, relative to globin expression prior to administration of the recombinant nucleic acid or the vector. In some embodiments, following administration of a recombinant nucleic acid or vector comprising a recombinant nucleic acid as described herein to a subject having a thalassemia, globin (e.g., gamma-globin) expression in the subject is increased about 5-8%, about 7-10%, about 9-12%, about 11-14%, about 13-16%, about 15-18%, about 17-20%, about 19-22%, or about 21-25%, relative to globin expression prior to administration of the recombinant nucleic acid or the vector. In some embodiments, following administration of a recombinant nucleic acid or vector comprising a recombinant nucleic acid as described herein to a subject having a thalassemia, globin (e.g., gamma-globin) expression in the subject is increased more than 25%, relative to globin expression prior to administration of the recombinant nucleic acid or the vector. In some embodiments, wherein fetal hemoglobin (e.g., gamma-globin) levels are about 13% (e.g., about 10-20%) of the total globin level in a subject (e.g., following administration of a recombinant nucleic acid or vector comprising a recombinant nucleic acid as described herein), one or more signs or symptoms of a thalassemia are alleviated or ameliorated in the subject.

In some embodiments, the subject has or is suspected of having a disease involving blood cells (e.g., a disease caused by a defect, such as a genetic mutation, in one or more blood cell types). Exemplary blood cells include T cell, B cells, dendritic cells, macrophages, monocytes, and hematopoietic stem cells. In some embodiments, the disease is a blood cell cancer, e.g., a leukemia (such as Acute lymphocytic leukemia, Acute myelogenous leukemia, Chronic lymphocytic leukemia, or Chronic myelogenous leukemia), lymphoma (such as Hodgkin lymphoma or non-Hodgkin lymphoma), or myeloma (such as multiple myeloma). Other exemplary diseases involving blood cells include anemia, hemophilia, myelodysplastic syndrome, sickle cell disease, thalassemia, deep vein thrombosis, von Willebrand disease, factor II, V, VII, X, or XII deficiency, Polycythemia vera, thrombocytopenia and Idiopathic thrombocytopenia purpura. Subjects having such diseases can be identified by the skilled practitioner according to methods known in the art, for example using one or more of: a complete blood count, platelet aggregation test, bleeding time test, genetic testing, and biomarker assays.

In some embodiments, a recombinant nucleic acid or vector comprising a recombinant nucleic acid as described herein is administered to a subject in combination with one or more additional therapeutic agents. Additional therapeutic agents may comprise, for example, anti-cancer or chemotherapeutic agents, antibodies, small molecules, and the like. In some embodiments, the subject has or is suspected of having more than one disease or condition. For example, in some embodiments, a subject may have or be suspected of having a hemoglobinopathy and a cancer.

In some embodiments, the method comprises contacting a cell with a recombinant nucleic acid of the disclosure or a vector comprising a recombinant nucleic acid of the disclosure, as described herein. In some embodiments, a cell disclosed herein is a cell isolated or derived from a subject. In some embodiments, a cell is a mammalian cell (e.g., a cell isolated or derived from a mammal). In some embodiments, the cell is a human cell, non-human primate cell, rat cell, or mouse cell. In some embodiments, a cell is in vitro. In some embodiments, a cell is ex vivo. In some embodiments, a cell is in vivo. In some embodiments, a cell is within a subject (e.g., within a tissue or organ of a subject). In some embodiments, a cell is a primary cell. In some embodiments, a cell is from a cell line (e.g., an immortalized cell line). In some embodiments a cell is a cancer cell or an immortalized cell.

Methods of contacting a cell may comprise, for example, contacting a cell in a culture with a recombinant nucleic acid of the disclosure or a vector comprising a recombinant nucleic acid of the disclosure, as described herein. In some embodiments, contacting a cell comprises adding a recombinant nucleic acid as described herein or a vector comprising a recombinant nucleic acid as described herein to the supernatant of a cell culture (e.g., a cell culture on a tissue culture plate or dish) or mixing a recombinant nucleic acid as described herein or a vector comprising a recombinant nucleic acid as described herein with a cell culture (e.g., a suspension cell culture). In some embodiments, contacting a cell comprises mixing a recombinant nucleic acid as described herein or a vector comprising a recombinant nucleic acid as described herein with another solution, such as a cell culture media, and incubating a cell with the mixture.

In some embodiments, contacting a recombinant nucleic acid as described herein or a vector comprising a recombinant nucleic acid as described herein comprises administering a recombinant nucleic acid as described herein or a vector comprising a recombinant nucleic acid as described herein to a subject or device in which the cell is located. In some embodiments, contacting a cell comprises injecting a recombinant nucleic acid as described herein or a vector comprising a recombinant nucleic acid as described herein into a subject in which the cell is located. In some embodiments, contacting a cell comprises administering a recombinant nucleic acid as described herein or a vector comprising a recombinant nucleic acid as described herein directly to a cell, or into or substantially adjacent to a tissue of a subject in which the cell is present.

TABLE 1
Exemplary sequences of the disclosure

| SEQ | | |
|---|---|---|
| ID NO | Description | Sequence |
| 1 | Target site | ATAAAAGCAACTGTTAGC |
| | within the | |
| | BCL11A | |
| | +58 kb GATA | |
| | binding motif | |
| 2 | Alternative | GTGATAAAAGCAACTGTT |
| | target site | |
| | within the | |
| | BCL11A | |
| | +58 kb GATA | |
| | binding motif | |
| 3 | 6ZF-DBD | LEPGEKPYKCPECGKSFSERSHLREHQRTHTGEKPYKCPECGKSFSTS |
| | amino acid | GSLVRHQRTHTGEKPYKCPECGKSFSTHLDLIRHQRTHTGEKPYKCPE |
| | sequence | CGKSFSQSGDLRRHQRTHTGEKPYKCPECGKSFSQRANLRAHQRTHTG |
| | capable of | EKPYKCPECGKSFSQKSSLIAHQRTHTGKKTS |
| | targeting a | Zinc finger motifs are underlined (SEQ ID NOs: 5-10) |
| | GATA motif in | |
| | a Bcl11A | |
| | enhancer | |
| 4 | Alternative | LEPGEKPYKCPECGKSFSTSGSLVRHQRTHTGEKPYKCPECGKSFSTH |
| | 6ZF-DBD | LDLIRHQRTHTGEKPYKCPECGKSFSQSGDLRRHQRTHTGEKPYKCPE |
| | amino acid | CGKSFSQRANLRAHQRTHTGEKPYKCPECGKSFSQKSSLIAHQRTHTG |
| | sequence | EKPYKCPECGKSFSRSDELVRHQRTHTGKKTS |
| | capable of | Zinc finger motifs are underlined (SEQ ID NOs: 11-16) |
| | targeting a | |
| | GATA motif in | |
| | a Bcl11A | |
| | enhancer | |
| 5 | Zinc Finger | ERSHIRE |
| | Motif 1-1 | |
| 6 | Zinc Finger | TSGSLVR |
| | Motif 1-2 | |
| 7 | Zinc Finger | THLDLIR |
| | Motif 1-3 | |
| 8 | Zinc Finger | QSGDLRR |
| | Motif 1-4 | |
| 9 | Zinc Finger | QRANLRA |
| | Motif 1-5 | |
| 10 | Zinc Finger | QKSSLIA |
| | Motif 1-6 | |
| 11 | Zinc Finger | TSGSLVR |
| | Motif 2-1 | |
| 12 | Zinc Finger | THLDLIR |
| | Motif 2-2 | |
| 13 | Zinc Finger | QSGDLRR |
| | Motif 2-3 | |
| 14 | Zinc Finger | QRANLRA |
| | Motif 2-4 | |
| 15 | Zinc Finger | QKSSLIA |
| | Motif 2-5 | |
| 16 | Zinc Finger | RSDELVR |
| | Motif 2-6 | |
| 17 | Wild-type | TTGCCCACTCCCTCTATGCGCGCTCGCTCGCTCGGTGGGGCCTGCGGA |
| | AAV6-ITR | CCAAAGGTCCGCAGACGGCAGAGCTCTGCTCTGCCGGCCCCACCGAGC |
| | | GAGCGAGCGCGCATAGAGGGAGTGGGCAACTCCATCACTAGGGGTA |
| 18 | AAV6-ITR | TTGCCCACTCCCTCTATGCGCGCTCGCTCGCTCGGTGGGGCCTGCGGA |
| | | CCAAAGGTCCGCAGACGGCAGAGCTCTGCTCTGCCGGCCCCACCGAGC |
| | | GAGCGAGCGCGCATAGAGGGAGTGGGCAACTCCATCACTAGGGGTAT |
| | | D sequence-19 end nucleotides-indicated by underlining |
| 19 | pscITR6- | cagcagctggcgtaatagcgaagaggcccgcaccgatcgcccttccca |
| | B19p6-6ZF- | acagttgcgcagcctgaatggcgaatggaattccagacgattgagcgt |
| | DBD-Krab- | caaaatgtaggtatttccatgagcgtttttcctgttgcaatggctggc |
| | 2021 | ggtaatattgttctggatattaccagcaaggccgatagtttgagttct |
| | | tctactcaggcaagtgatgttattactaatcaaagaagtattgcgaca |
| | | acggttaatttgcgtgatggacagactcttttactcggtggcctcact |
| | | gattataaaaacacttctcaggattctggcgtaccgttcctgtctaaa |
| | | atccctttaatcggcctcctgtttagctcccgctctgattctaacgag |
| | | gaaagcacgttatacgtgctcgtcaaagcaaccatagtacgcgccctg |
| | | tagcggcgcattaagcgcggcgggtgtggtggttacgcgcagcgtgac |
| | | cgctacacttgccagcgccctagcgcccgctcctttcgctttcttccc |
| | | ttcctttctcgccacgttcgccggctttccccgtcaagctctaaatcg |
| | | ggggctccctttagggttccgatttagtgctttacggcacctcgaccc |
| | | caaaaaacttgattagggtgatggttcacgtagtgggccatcgccctg |
| | | atagacggtttttcgccctttgacgttggagtccacgttctttaatag |
| | | tggactcttgttccaaactggaacaacactcaaccctatctcggtcta |
| | | ttcttttgatttataagggattttgccgatttcggcctattggttaaa |
| | | aaatgagctgatttaacaaaaatttaacgcgaattttaacaaaatatt |
| | | aacgtttacaatttaaatatttgcttatacaatcttcctgtttttggg |
| | | gcttttctgattatcaaccggggtacatatgattgacatgctagtttt |
| | | acgattaccgttcatcgccttgcccactccctctatgcgcgctcgctc |
| | | gctcggtggggcctgcggaccaaaggtccgcagacggcagagctctgc |
| | | tctgccggccccaccgagcgagcgagcgcgcatagagggagtgggcaa |
| | | aattcacgcgtggatctgaattcaattcacgcgtGGTACggtacctgt |
| | | gacgtcacaggaaatgacgtaattgtccgccatcttgtaccggaagtc |
| | | ccgcctaccggcggcgaccggcggcatctgatttggtgtcttctttta |
| | | aattttagcgggcttttttcccgccttatgcaaatgggcagccatttt |
| | | aagtgttttactataattttattggttagttttgtaacggttaaaatg |
| | | ggcggagcgtaggcggggactacagtatatatagcacggtactgccgc |
| | | agctctttctttctgggctgctttttcctggactttcttgctgttttt |
| | | tgtgagctaactaacaggtatttatactacttgttaacatcctaacac |
| | | gcgtCTCGAGGAATTCATGCCGAAAAAAAAACGCAAAGTGCTAGAGCC |
| | | CGGGGAGAAACCCTATAAATGTCCCGAGTGCGGCAAGTCCTTCTCGGA |
| | | GCGGTCCCACCTGCGGGAGCATCAGCGTACCCACACGGGCGAAAAGCC |
| | | GTACAAATGCCCAGAATGCGGTAAATCCTTCAGCACCTCCGGCTCCCT |
| | | GGTGCGGCATCAACGGACGCATACAGGAGAGAAGCCATACAAATGTCC |
| | | CGAATGTGGGAAGAGTTTTAGTACCCACCTGGACCTGATCCGGCACCA |
| | | ACGAACTCACACCGGTGAGAAGCCCTATAAGTGTCCGGAGTGTGGCAA |
| | | GTCGTTCTCGCAGTCCGGCGACCTGCGGCGGCACCAGCGCACTCATAC |
| | | TGGTGAGAAGCCCTATAAATGCCCGGAATGTGGGAAGTCGTTTAGTCA |
| | | GCGGGCCAACCTGCGGGCCCATCAACGTACCCACACCGGGGAAAAACC |
| | | TTATAAGTGCCCAGAGTGCGGTAAAAGCTTCAGCCAGAAGTCCTCCCT |
| | | GATCGCCCACCAGCGCACTCACACCGGCAAAAAAACCAGCAGGCCTGA |
| | | TGCTAAGTCACTGACTGCCTGGTCCCGGACACTGGTGACCTTCAAGGA |
| | | TGTGTTTGTGGACTTCACCAGGGAGGAGTGGAAGCTGCTGGACACTGC |
| | | TCAGCAGATCCTGTACAGAAATGTGATGCTGGAGAACTATAAGAACCT |
| | | GGTTTCCTTGGGTTATCAGCTTACTAAGCCAGATGTGATCCTCCGGTT |
| | | GGAGAAGGGAGAAGAGCCCTGGCTGGTGGAGAGAGAAATTCACCAAGA |
| | | GACCCATCCTGATTCAGAGACTGCATTTGAAATCAAATCATCAGTTAG |
| | | ATCTAGGCCTGATTATAAAGATGATGATGATAAAGATTATAAAGATGA |
| | | TGATGATAAAGATTATAAAGATGATGATGATAAATGAagcttatcgat |
| | | accgtcgactagagctcgctgatcagcctcgactgtgccttctagttg |
| | | ccagccatctgttgtttgcccctcccccgtgccttccttgaccctgga |
| | | aggtgccactcccactgtcctttcctaataaaatgaggaaattgcatc |
| | | gcattgtctgagtaggtgtcattctattctggggggtggggtggggca |
| | | ggacagcaagggggaggattgggaagacaatagcaggcatgctgggga |
| | | gagatcgatctggaatacccctagtgatggagttgcccactccctcta |
| | | tgcgcgctcgctcgctcggtggggccggcagagcagagctctgccgtc |
| | | tgcggacctttggtccgcaggccccaccgagcgagcgagcgcgcatag |
| | | agggagtgggcaacccccccccccccccccctgcatgcaggcgattct |
| | | cttgtttgctccagactctcaggcaatgacctgatagcctttgtagag |
| | | acctctcaaaaatagctaccctctccggcatgaatttatcagctagaa |
| | | cggttgaatatcatattgatggtgatttgactgtctccggcctttctc |
| | | acccgtttgaatctttacctacacattactcaggcattgcatttaaaa |
| | | tatatgagggttctaaaaatttttatccttgcgttgaaataaaggctt |
| | | ctcccgcaaaagtattacagggtcataatgtttttggtacaaccgatt |
| | | tagctttatgctctgaggctttattgcttaattttgctaattctttgc |
| | | cttgcctgtatgatttattggatgttggaattcctgatgcggtatttt |
| | | ctccttacgcatctgtgcggtatttcacaccgcatatggtgcactctc |
| | | agtacaatctgctctgatgccgcatagttaagccagccccgacacccg |
| | | ccaacacccgctgacgcgccctgacgggcttgtctgctcccggcatcc |
| | | gcttacagacaagctgtgaccgtctccgggagctgcatgtgtcagagg |
| | | ttttcaccgtcatcaccgaaacgcgcgagacgaaagggcctcgtgata |
| | | cgcctatttttataggttaatgtcatgataataatggtttcttagacg |
| | | tcaggtggcacttttcggggaaatgtgcgcggaacccctatttgttta |
| | | tttttctaaatacattcaaatatgtatccgctcatgagacaataaccc |
| | | tgataaatgcttcaataatattgaaaaaggaagagtatgagtattcaa |
| | | catttccgtgtcgcccttattcccttttttgcggcattttgccttcct |
| | | gtttttgctcacccagaaacgctggtgaaagtaaaagatgctgaagat |
| | | cagttgggtgcacgagtgggttacatcgaactggatctcaacagcggt |
| | | aagatccttgagagttttcgccccgaagaacgttttccaatgatgagc |
| | | acttttaaagttctgctatgtggcgcggtattatcccgtattgacgcc |
| | | gggcaagagcaactcggtcgccgcatacactattctcagaatgacttg |
| | | gttgagtactcaccagtcacagaaaagcatcttacggatggcatgaca |
| | | gtaagagaattatgcagtgctgccataaccatgagtgataacactgcg |
| | | gccaacttacttctgacaacgatcggaggaccgaaggagctaaccgct |
| | | tttttgcacaacatgggggatcatgtaactcgccttgatcgttgggaa |
| | | ccggagctgaatgaagccataccaaacgacgagcgtgacaccacgatg |
| | | cctgtagcaatggcaacaacgttgcgcaaactattaactggcgaacta |
| | | cttactctagcttcccggcaacaattaatagactggatggaggcggat |
| | | aaagttgcaggaccacttctgcgctcggcccttccggctggctggttt |
| | | attgctgataaatctggagccggtgagcgtgggtctcgcggtatcatt |
| | | gcagcactggggccagatggtaagccctcccgtatcgtagttatctac |
| | | acgacggggagtcaggcaactatggatgaacgaaatagacagatcgct |
| | | gagataggtgcctcactgattaagcattggtaactgtcagaccaagtt |
| | | tactcatatatactttagattgatttaaaacttcatttttaatttaaa |
| | | aggatctaggtgaagatcctttttgataatctcatgaccaaaatccct |
| | | taacgtgagttttcgttccactgagcgtcagaccccgtagaaaagatc |
| | | aaaggatcttcttgagatcctttttttctgcgcgtaatctgctgcttg |
| | | caaacaaaaaaaccaccgctaccagcggtggtttgtttgccggatcaa |
| | | gagctaccaactctttttccgaaggtaactggcttcagcagagcgcag |
| | | ataccaaatactgtccttctagtgtagccgtagttaggccaccacttc |
| | | aagaactctgtagcaccgcctacatacctcgctctgctaatcctgtta |
| | | ccagtggctgctgccagtggcgataagtcgtgtcttaccgggttggac |
| | | tcaagacgatagttaccggataaggcgcagcggtcgggctgaacgggg |
| | | ggttcgtgcacacagcccagcttggagcgaacgacctacaccgaactg |
| | | agatacctacagcgtgagctatgagaaagcgccacgcttcccgaaggg |
| | | agaaaggcggacaggtatccggtaagcggcagggtcggaacaggagag |
| | | cgcacgagggagcttccagggggaaacgcctggtatctttatagtcct |
| | | gtcgggtttcgccacctctgacttgagcgtcgatttttgtgatgctcg |
| | | tcaggggggcggagcctatggaaaaacgccagcaacgCggccttttta |
| | | cggttcctggccttttgctggccttttgctcacatgttctttcctgcg |
| | | ttatcccctgattctgtggataaccgtattaccgcctttgagtgagct |
| | | gataccgctcgccgcagccgaacgaccgagcgcagcgagtcagtgagc |
| | | gaggaagcggaagagcgcccaatacgcaaaccgcctctccccgcgcgt |
| | | tggccgattcattaatg |
| 20 | Polynucleotide | GACAAGCTTCACCATCATCACCACCATCACCACCATCACCATCACGAA |
| | encoding 6ZF- | TTCCTCGAGCTAGAGCCCGGGGAGAAACCCTATAAATGTCCCGAGTGC |
| | DBD protein | GGCAAGTCCTTCTCGGAGCGGTCCCACCTGCGGGAGCATCAGCGTACC |
| | capable of | CACACGGGCGAAAAGCCGTACAAATGCCCAGAATGCGGTAAATCCTTC |
| | targeting a | AGCACCTCCGGCTCCCTGGTGCGGCATCAACGGACGCATACAGGAGAG |
| | GATA motif in | AAGCCATACAAATGTCCCGAATGTGGGAAGAGTTTTAGTACCCACCTG |
| | a Bcl11A | GACCTGATCCGGCACCAACGAACTCACACCGGTGAGAAGCCCTATAAG |
| | enhancer | TGTCCGGAGTGTGGCAAGTCGTTCTCGCAGTCCGGCGACCTGCGGCGG |
| | | CACCAGCGCACTCATACTGGTGAGAAGCCCTATAAATGCCCGGAATGT |
| | | GGGAAGTCGTTTAGTCAGCGGGCCAACCTGCGGGCCCATCAACGTACC |
| | | CACACCGGGGAAAAACCTTATAAGTGCCCAGAGTGCGGTAAAAGCTTC |
| | | AGCCAGAAGTCCTCCCTGATCGCCCACCAGCGCACTCACACCGGCAAA |
| | | AAAACCAGCAGGCCTGTC |
| 21 | Polynucleotide | TGCCGAAAAAAAAACGCAAAGTGCTAGAGCCCGGGGAGAAACCCTATA |
| | encoding 8ZF- | AGTGTCCGGAGTGTGGCAAGTCGTTCTCGCGGGCCGACAACCTGACCG |
| | DBD protein | AGCATCAGCGTACCCACACGGGCGAAAAGCCGTACAAATGCCCAGAAT |
| | capable of | GCGGTAAATCCTTCAGCACCTCCCACTCCCTGACCGAGCATCAACGGA |
| | targeting a | CGCATACAGGAGAGAAGCCATACAAATGTCCCGAATGTGGGAAGAGTT |
| | gamma-globin | TTAGTCAGCGGGCCAACCTGCGGGCCCACCAGCGCACTCACACCGGCG |
| | promoter | AAAAACCGTATAAGTGTCCCGAATGCGGAAAGTCATTTTCGCAGAACT |
| | | CCACCCTGACCGAGCACCAGCGGACACATACCGGTGAGAAGCCCTATA |
| | | AGTGTCCGGAGTGTGGCAAGTCGTTCTCCGACCCCGGCCACCTGGTGC |
| | | GGCATCAGCGTACCCACACGGGCGAAAAGCCGTACAAATGCCCAGAAT |
| | | GCGGTAAATCCTTCAGCACCACCGGCAACCTGACCGTGCATCAACGGA |
| | | CGCATACAGGAGAGAAGCCATACAAATGTCCCGAATGTGGGAAGAGTT |
| | | TTAGTACCTCCGGCAACCTGGTGCGGCACCAGCGCACTCACACCGGCG |
| | | AAAAACCGTATAAGTGTCCCGAATGCGGAAAGTCATTTTCGCAGCTGG |
| | | CCCACCTGCGGGCCCACCAACGCACGCACACCGGCAAAAAAACCAGCG |
| | | ATTATAAAGATGATGATGATAAAGATTATAAAGATGATGATGATAAAG |
| | | ATTATAAAGATGATGATGATAAATGA |
| 22 | B19p6 | CCAACCCTAATTCCGGAAGTCCCGCCCACCGGAAGTGACGTCACAGGA |
| | promoter | AATGACGTCACAGGAAATGACGTAATTGTCCGCCATCTTGTACCGGAA |
| | | GTCCCGCCTACCGGCGGCGACCGGCGGCATCTGATTTGGTGTCTTCTT |
| | | TTAAATTTTAGCGGGCTTTTTTCCCGCCTTATGCAAATGGGCAGCCAT |
| | | TTTAAGTGTTTTACTATAATTTTATTGGTTAGTTTTGTAACGGTTAAA |
| | | ATGGGCGGAGCGTAGGCGGGGACTACAGTATATATAGCACGGCACTGC |
| | | CGCAGCTCTTTCTTTCTGGGCTGCTTTTTCCTGGACTTTCTTGCTGTT |
| | | TTTTGTGAGCTAACTAACAGGTATTTATACTACTTGTTAACATACTAA |
| 23 | 8ZF-DBD | LEPGEKPYKCPECGKSFSRADNLTEHQRTHTGEKPYKCPECGKSFSTS |
| | amino acid | HSLTEHQRTHIGEKPYKCPECGKSFSQRANLRAHQRTHIGEKPYKCPE |
| | sequence | CGKSFSQNSTLTEHQRTHTGEKPYKCPECGKSFSDPGHLVRHQRTHTG |
| | capable of | EKPYKCPECGKSFSTTGNLTVHQRTHTGEKPYKCPECGKSFSTSGNLV |
| | targeting a | RHQRTHTGEKPYKCPECGKSFSQLAHLRAHQRTHTGKKTS |
| | gamma-globin | Zinc finger motifs are underlined (SEQ ID NOs: 24-31) |
| | promoter | |
| 24 | Zinc Finger | RADNLTE |
| | Motif 3-1 | |
| 25 | Zinc Finger | TSHSLTE |
| | Motif 3-2 | |
| 26 | Zinc Finger | QRANLRA |
| | Motif 3-3 | |
| 27 | Zinc Finger | QNSTLTE |
| | Motif 3-4 | |
| 28 | Zinc Finger | DPGHLVR |
| | Motif 3-5 | |
| 29 | Zinc Finger | TTGNLTV |
| | Motif 3-6 | |
| 30 | Zinc Finger | TSGNLVR |
| | Motif 3-7 | |
| 31 | Zinc Finger | QLAHLRA |
| | Motif 3-8 | |

### DEFINITIONS

Unless otherwise defined herein, all scientific and technical terms used in connection with the present disclosure shall have the meanings that are commonly understood by those of ordinary skill in the art. The meaning and scope of the terms are clear; however, in the event of any latent ambiguity, definitions provided herein take precedent over any dictionary or extrinsic definition. Further, unless otherwise required by context, singular terms shall include pluralities and plural terms shall include the singular. In this disclosure, the use of “or” means “and/or” unless stated otherwise. Furthermore, the use of the term “including,” as well as other forms, such as “includes” and “included,” is not limiting. Things described as “including” or “comprising” can also be configured as “consisting of” or similar language. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

These and other aspects of the application are illustrated by the following non-limiting examples.

### Examples

### Example 1: Selection of Zinc Finger DNA-Binding Domains (ZF-DBDs) Based on Affinity for Target Sites within the BCL11A+58 kb GATA Binding Motif

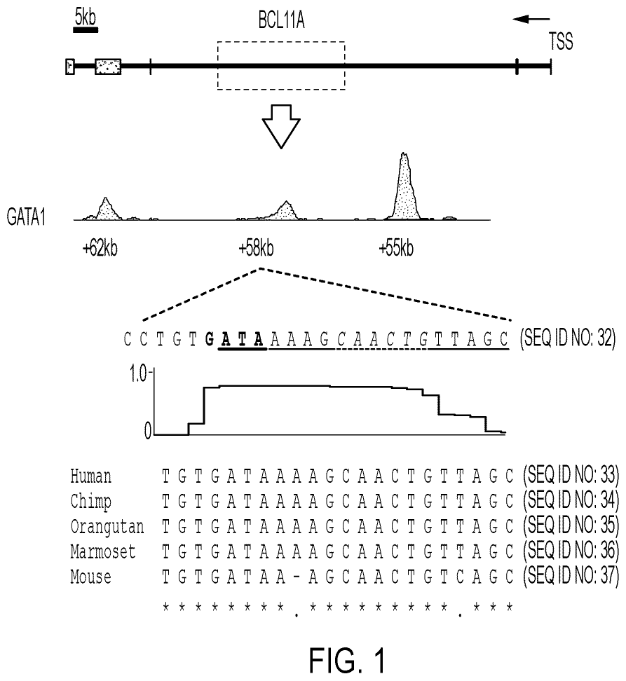

The binding affinities of two different ZF-DBD proteins (SEQ ID NOs: 3 and 4) for two target sites within the BCL11A+58 kb GATA binding motif (SEQ ID NOs: 1 and 2, respectively; see FIG. 1) were assessed. A DNA-binding assay was performed on each of six zinc finger motifs (SEQ ID NOs: 5-10) isolated from SEQ ID NO: 3 (results shown in FIG. 2A), and for each of six zinc finger motifs (SEQ ID NOs: 11-16) isolated from SEQ ID NO: 4 (results shown in FIG. 2B). The results of FIG. 2A show a higher binding specificity for the first and last zinc finger motifs (SEQ ID NOs: 5 and 10) isolated from SEQ ID NO: 3 than the zinc finger motifs (SEQ ID NOs: 11-16) isolated from SEQ ID NO: 4 (FIG. 2B). Hence, the 6ZF-DBD of SEQ ID NO: 3, targeting the GATA motif in the Bcl11A enhancer shown in SEQ ID NO: 1, was selected to pursue further experiments.

### Example 2: Assessment of the Binding Affinity of the Selected 6ZFN-DBD to the Target Site within the BCL11A+58 kb GATA Binding Motif

An Electrophoretic Mobility Shift DNA-binding assay (EMSA) was used to determine the binding affinity of the selected 6ZFN-DBD (amino acid sequence shown in SEQ ID NO: 3; nucleic acid sequence shown in SEQ ID NO: 20) to the GATA motif in the Bcl11A enhancer shown in SEQ ID NO: 1. Results showed that the dissociation constant (K_{d}) was about 53 nM (52.8 ±1.4 nM; see FIG. 3). This dissociation constant compares well relative to dissociation constants of other ZF-DBDs targeting gamma-globin promoters, which effected substantial reactivation of the gamma-globin function, but only exhibited Kds of 45 and 23 nM for their respective targets (see, e.g., Barrow, et al. (2012), Neutralizing the function of a β-globin-associated cis-regulatory DNA element using an artificial zinc finger DNA-binding domain, PNAS 109 (44): 17948-953; Barrow, et al. (2014), Dissecting the function of the adult β-globin downstream promoter region using an artificial zinc finger DNA-binding domain, Nucl Acid Res, 42 (7): 4363-74; Hossain, et al. (2015), Artificial Zinc Finger DNA Binding Domains: Versatile Tools for Genome Engineering and Modulation of Gene Expression, J Cell Biochem, 116 (11) 2435-444; Hossain, et al. (2016), Activation of Fetal γ-globin Gene Expression via Direct Protein Delivery of Synthetic Zinc-finger DNA-Binding Domains, Mol Ther Nucl Acid, 5 (E378); Li, et al. (2018), Fetal hemoglobin induction in sickle erythroid progenitors using a synthetic zinc finger DNA-binding domain, Haematalogica, 103 (9): e384-87).

The results observed for the 6ZF-DBD targeting the BCL11A+58 kb GATA binding motif (amino acid sequence shown in SEQ ID NO: 3; nucleic acid sequence shown in SEQ ID NO: 20) indicate that the 6ZFN-DBD is likely to affect the expression of BCL11A through its interaction with its target (the GATA motif in the Bcl11A enhancer shown in SEQ ID NO: 1).

### Example 3: Expression of Gamma-Globin in Cells Transduced with an rAAV Vector Encoding the Selected 6ZF-DBD which Targets the BCL11A+58 kb GATA Binding Motif

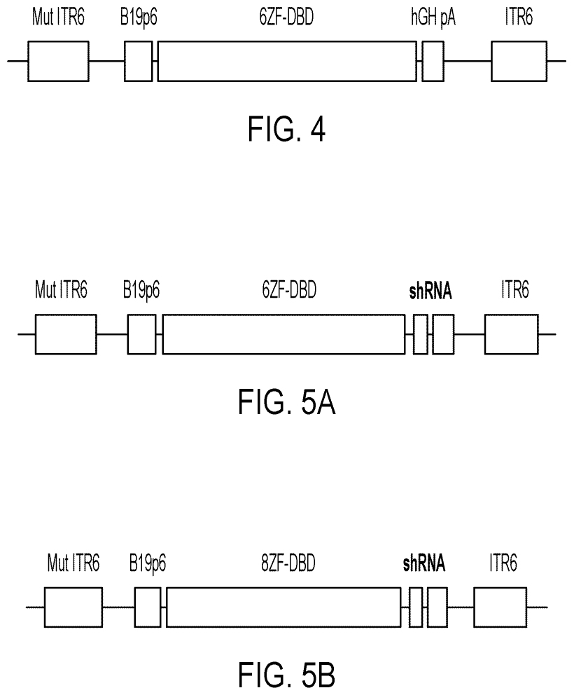

Peripheral blood mononuclear cells (PBMCs) are isolated from sickle cell patients and are used to induce differentiation along the erythroid lineage. On day 8 of differentiation, cells are transduced with an AAV vector (SEQ ID NO: 19) comprising: (a) a B19p6 promoter (e.g., SEQ ID NO: 23), (b) a nucleic acid encoding the selected 6ZF-DBD targeting the BCL11A+58 kb GATA binding motif (SEQ ID NO: 20), and (c) a human growth hormone (hGH) or bovine growth hormone (bGH) poly A tail, flanked by a mutITR6 ITR (e.g., SEQ ID NO: 18) on the 5′ end of the vector and a wild-type AAV6 ITR (SEQ ID NO: 17) on the 3′ end (see FIG. 4), at concentrations of 2×10⁴ vgs/cell or 2×10⁵ vgs/cell.

Localization of the encoded 6ZF-DBD targeting the BCL11A+58 kb GATA binding motif in the PBMC cytoplasm and nucleus is assessed by isolating proteins from the cytoplasm and nucleus as previously described (Hossain, et al. (2016), Activation of Fetal γ-globin Gene Expression via Direct Protein Delivery of Synthetic Zinc-finger DNA-Binding Domains, Mol Ther Nucl Acid, 5 (E378)). Western Blot analysis is conducted using antibodies specific for the 6ZF-DBD protein. Specific interaction of the 6ZF-DBD with its target binding site (the GATA motif in the Bcl11A enhancer shown in SEQ ID NO: 1) is measured by purifying DNA collected from transduced PBMC cells. The purified DNA is then analyzed by quantitative PCR (qPCR) using primers specific for the target site within the BCL11A+58 kb GATA binding motif.

To measure transcription rates of the gamma-globin and beta-globin genes, reverse transcriptase-qPCR (RTqPCR) is conducted using RNA isolated from sickle erythroid progenitors after 48 h of treatment (day 10) in culture and primers specific to gamma-globin and beta-globin. Increased transcription of the gamma-globin gene, but not beta-globin gene, is observed in cells transduced with the AAV vector (SEQ ID NO: 19), as compared to the control group not transduced. Additionally, cells transduced with higher vector genome counts of the AAV vector (SEQ ID NO: 19) show higher transcription levels of the gamma-globin gene, as compared to cells transduced with lower vector genome counts.

These results indicate that the AAV vector (SEQ ID NO: 19) facilitates 6ZFN-DBD expression in transduced cells, and that the interaction of the 6ZF-DBD targeting the BCL11A+58 kb GATA binding motif (SEQ ID NO: 3) with its target (SEQ ID NO: 1) increases gamma-globin expression.

### Example 4: Expression of Gamma-Globin in Cells Transduced with AAV Vectors Encoding ZF-DBDs and a shRNA Targeting BCL11A mRNA

Erythroid-differentiated primary human CD34+ cells obtained as described in Example 3 are transduced with AAV vectors comprising: (a) a B19p6 promoter (e.g., SEQ ID NO: 23), (b) a coding sequence for either the selected 6ZF-DBD targeting the BCL11A+58 kb GATA binding motif (SEQ ID NO: 20) or an 8ZF-DBD targeting the gamma-globin promoter (SEQ ID NO: 21), (c) a shRNA sequence targeting BCL11A mRNA, (d) optionally, a polynucleotide encoding a fluorescent protein (e.g., Venus, RFP, GFP), and (e) a human growth hormone (hGH) or bovine growth hormone (bGH) polyA tail, flanked by a mutITR6 ITR (e.g., SEQ ID NO: 18) on the 5′ end of the vector and a wild-type AAV6 ITR (SEQ ID NO: 17) on the 3′ end (FIG. 5A shows the 6ZF-DBD+shRNA construct; FIG. 5B shows the 8ZF-DBD+shRNA construct). Proper targeting of the vector is verified through fluorescence microscopy. Expression of gamma-globin is measured using real-time polymerase chain reaction (RT-PCR), iso-electric gel electrophoresis, or high-performance liquid chromatography (HPLC). Cells transduced with the vectors show a significant, dose-dependent increase in expression of gamma-globin, compared to a control group not transduced.

### Example 5: Assessment of Recombinant Nucleic Acid Encoding 6ZF-DBD Targeting the BCL11A+58 kb GATA Binding Motif in Improving Gamma-Globin Expression and Decreasing Sickling In Vivo

Subjects (e.g., mice, non-human primates, humans) suffering from sickle cell disease are administered a vector as described in Examples 4 and 5, and as shown in FIGS. 4, 5A, and 5B. The vectors are administered via intravenous injection or directly to the bone marrow of the subject. Ten days after administration of the vector, peripheral blood mononuclear cells (PBMCs) are isolated from the subject and stained with FITC-labeled anti-gamma-globin antibody. The stained PBMC cells are analyzed by flow cytometry to determine counts of cells expressing gamma-globin. Cell counts for the gamma-globin expressing PBMCs post-administration are compared to those of gamma-globin-expressing PBMCs obtained from the same subject one day prior to administration of the vector. Western blot analysis is further conducted to confirm results. Results show that gamma-globin expression is increased in subjects who were administered the vectors, as compared to the subject's baseline prior to vector administration and to subjects in a control group who did not receive treatment.

Erythroid progenitor cells (or red blood cells) are isolated from the subjects who were administered a vector, and incubated under normoxic (20% (2) or hypoxic (1% (2) conditions. The number of sickle cells is quantified under light microscopy. Results show that under hypoxic conditions the amount of cell sickling is substantially lower in subjects injected with the vector of the disclosure, compared to baseline, as compared to the subject's baseline prior to vector administration and to subjects in a control group who did not receive treatment,

These results indicate that the AAV vectors described herein allow successful delivery and expression of the 6ZF-DBD or 8ZF-DBD proteins (targeting the BCL11A+58 kb GATA binding motif and the gamma-globin promoter, respectively), and in some embodiments of an shRNA sequence targeting BCL11A mRNA, in a way that quantitatively affects gamma-globin expression levels and sickling in vivo. As a result of administration of a vector of the disclosure, gamma globin levels are increased in the subject, and one or more signs or symptoms of the subject's disease or disorder (e.g., sickle cell, β-thalassemia) is alleviated or obviated.

### Example 6: Recombinant AAV6-B19p6-8ZFN Vector-Mediated Reactivation of Fetal Hemoglobin Expression in Primary Human CD34+ Cells Following Erythroid-Differentiation In Vitro

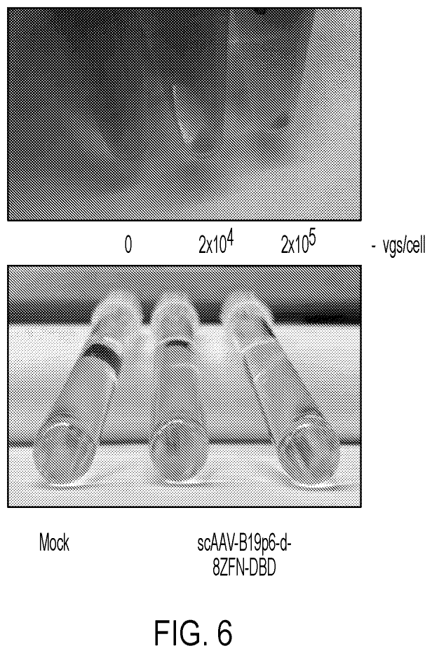

One million human CD34⁺ cells (StemCell Technologies) were transduced for 2 hrs at 37 °C. The cells were either mock infected or infected with AAV6-B19p6-8ZFN (SEQ ID NO: 38; 2×10⁴ vgs/cell) or AAV6-B19p6-8ZFN (SEQ ID NO: 38; 2×10⁵ vgs/cell) (FIG. 6). Cells were cultured in serum-free medium (StemSpan SFEM II Kit) for 7 days followed by erythroid-differentiation for an additional 7 days using STEMdiff™ Hematopoietic Kit, and cell pellets were visualized before and after rinsing with PBS.

Following transduction, culture, erythroid-differentiation, and visualization, it was observed that the cells infected with AAV6-B19p6-8ZFN (SEQ ID NO: 38; 2×10⁴ vgs/cell) and AAV6-B19p6-8ZFN (SEQ ID NO: 38; 2×10⁵ vgs/cell) displayed reactivation of fetal hemoglobin. This data demonstrates that fetal hemoglobin levels can be increased using an rAAV vector construct of the present disclosure (AAV6-B19p6-8ZFN; SEQ ID NO: 38), and without the use of a nuclease.

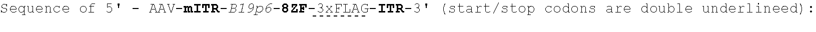

(SEQ ID NO: 38)
5′-ggccactccctctctgcgcgctcgctcgctcactgaggccgcccgggcaaagcccgggcgtcgggcgacctttggtcgcccg
gcctcagtgagcgagcgagcgcgcagagagggagtggccaaggtacctgtgacgtcacaggaaatgacgtaattgtccgccatctt
gtaccggaagtcccgcctaccggcggcgaccggcggcatctgatttggtgtcttcttttaaattttagcgggcttttttcccgccttatgcaa
atgggcagccattttaagtgttttactataattttattggttagttttgtaacggttaaaatgggcggagcgtaggcggggactacagtatat
atagcacggtactgccgcagctctttctttctgggctgctttttcctggactttcttgctgtttttgtgagctaactaacaggtatttatactactt
gttaacatcctaacacgcgtGAATTCATGCCGAAAAAAAAACGCAAAGTGCTAGAGCCCGGGG
AGAAACCCTATAAGTGTCCGGAGTGTGGCAAGTCGTTCTCGCGGGCCGACAACCTG
ACCGAGCATCAGCGTACCCACGGGCGAAAAGCCGTACAAATGCCCAGAATGCGG
TAAATCCTTCAGCACCTCCCACTCCCTGACCGAGCATCAACGGACGCATACAGGAGA
GAAGCCATACAAATGTCCCGAATGTGGGAAGAGTTTTAGTCAGCGGGCCAACCTGC
GGGCCCACCAGCGCACTCACACCGGCGAAAAACCGTATAAGTGTCCCGAATGCGGA
AAGTCATTTTCGCAGAACTCCACCCTGACCGAGCACCAGCGGACACATACCGGTGA
GAAGCCCTATAAGTGTCCGGAGTGTGGCAAGTCGTTCTCCGACCCCGGCCACCTGGT
GCGGCATCAGCGTACCCACACGGGCGAAAAGCCGTACAAATGCCCAGAATGCGGTA
AATCCTTCAGCAACCACCGGCAACCTGACCGTGCATCAACGGACGCATACAGGAGAG
AAGCCATACAAATGTCCCGAATGTGGGAAGAGTTTTAGTACCTCCGGCAACCTGGTG
CGGCACCAGCGCACTCACACCGGCGAAAAACCGTATAAGTGTCCCGAATGCGGAAA
GTCATTTTCGCAGCTGGCCCACCTGCGGGCCCACCAACGCACGCACAACCGGCAAAA

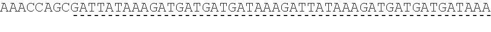

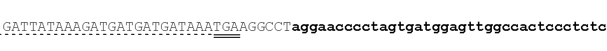

tgcgcgctcgctcgctcactgaggccgggcgaccaaaggtcgcccgacgcccgggctttgcccgggcggcctcagtgagcgagcg
agcgcgcagagagggagtggcc-3′

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A recombinant nucleic acid comprising a polynucleotide encoding a zinc finger DNA binding domain (ZF-DBD) that is capable of targeting a GATA motif in a Bcl11A enhancer.

2. The recombinant nucleic acid of claim 1, wherein the ZF-DBD is encoded by a polynucleotide having at least 90%, at least 95%, at least 98%, or at least 99% sequence identity to the nucleic acid sequence of SEQ ID NO: 20.

3. The recombinant nucleic acid of claim 1, wherein the ZF-DBD is encoded by a polynucleotide having a nucleic acid sequence as shown in SEQ ID NO: 20.

4. The recombinant nucleic acid of any one of claims 1-3, wherein the encoded ZF-DBD comprises a polypeptide having at least 90%, at least 95%, at least 98%, or at least 99% sequence identity to the nucleic acid sequence of SEQ ID NO: 3.

5. The recombinant nucleic acid of any one of claims 1-3, wherein the encoded ZF-DBD comprises a polypeptide having the sequence as shown in SEQ ID NO: 3.

6. A vector comprising the recombinant nucleic acid according to any one of claims 1-5.

7. The vector of claim 6, wherein the vector comprises a recombinant adeno-associated virus (rAAV) vector.

8. The vector of claim 6 or claim 7, further comprising a polynucleotide encoding a regulatory element.

9. The vector of claim 8, wherein the regulatory element comprises a human parvovirus B19 promoter at map unit 6 (B19p6 promoter).

10. The vector of any one of claims 6-9, further comprising one or more AAV inverted terminal repeats (ITRs).

11. The vector of claim 10, wherein the vector comprises two AAV ITRs.

12. The vector of claim 10 or claim 11, wherein the AAV ITR(s) are naturally-occurring AAV ITRs.

13. The vector of claim 10 or claim 11, wherein the AAV ITR(s) are synthetic AAV ITRs.

14. The vector of any one of claims 10-13, wherein the AAV ITR(s) are from AAV serotype 6 (AAV6).

15. The vector of any one of claims 6-14, further comprising a polyadenylation (pA) signal.

16. The vector of claim 15, wherein the pA signal comprises a bovine growth hormone pA (BGH pA) or a human growth hormone pA (HGH pA) signal.

17. The vector of any one of claims 6-16, further comprising a polynucleotide encoding a short hairpin RNA (shRNA) that is capable of targeting a Bcl11A mRNA transcript.

18. The vector of any one of claims 7-17, wherein the rAAV vector is a recombinant self-complimentary AAV (scAAV) vector.

19. The vector of any one of claims 7-18, wherein the rAAV vector is a recombinant AAV serotype 6 (AAV6) vector.

20. A recombinant nucleic acid comprising a polynucleotide encoding a zinc finger DNA binding domain (ZF-DBD) and a short hairpin RNA (shRNA), wherein the ZF-DBD is capable of targeting a gamma-globin promoter and the shRNA is capable of targeting a Bcl11A mRNA transcript.

21. The recombinant nucleic acid of claim 20, wherein the ZF-DBD is encoded by a polynucleotide having at least 90%, at least 95%, at least 98%, or at least 99% sequence identity to the nucleic acid sequence of SEQ ID NO: 21.

22. The recombinant nucleic acid of claim 20, wherein the ZF-DBD is encoded by a polynucleotide having a nucleic acid sequence as shown in SEQ ID NO: 21.

23. The recombinant nucleic acid of any one of claims 20-22, wherein the encoded ZF-DBD comprises a polypeptide having at least 90%, at least 95%, at least 98%, or at least 99% sequence identity to the amino acid sequence of SEQ ID NO: 23.

24. The recombinant nucleic acid of any one of claims 20-22, wherein the encoded ZF-DBD comprises a polypeptide having an amino acid as shown in SEQ ID NO: 23.

25. A vector comprising the recombinant nucleic acid according to any one of claims 20-24.

26. The vector of claim 25, wherein the vector comprises a recombinant adeno-associated virus (rAAV) vector.

27. The vector of claim 25 or claim 26, further comprising a polynucleotide encoding a regulatory element.

28. The vector of claim 27, wherein the regulatory element comprises a human parvovirus B19 promoter at map unit 6 (B19p6 promoter).

29. The vector of any one of claims 25-28, further comprising one or more AAV inverted terminal repeats (ITRs).

30. The vector of claim 29, wherein the vector comprises two AAV ITRs.

31. The vector of claim 29 or claim 30, wherein the AAV ITR(s) are naturally-occurring AAV ITRs.

32. The vector of claim 29 or claim 30, wherein the AAV ITR(s) are synthetic AAV ITRs.

33. The vector of any one of claims 29-32, wherein the AAV ITR(s) are from AAV serotype 6 (AAV6).

34. The vector of any one of claims 25-33, further comprising a polyadenylation (pA) signal.

35. The vector of claim 34, wherein the pA signal comprises a bovine growth hormone pA (BGH pA) or a human growth hormone pA (HGH pA) signal.

36. A method of promoting globin expression, the method comprising administering the recombinant nucleic acid according to any one of claim 1-5 or 20-24, or the vector according to any one of claim 6-19 or 25-35 to a subject.

37. The method of claim 36, wherein the subject has or is suspected of having β-thalassemia.

38. The method of claim 36, wherein the subject has or is suspected of having sickle cell disease.

39. The method of any one of claims 36-38, wherein the subject is a human, non-human primate, non-primate mammal, or mouse subject.

40. The method of any one of claims 36-39, wherein the recombinant nucleic acid or vector is administered intramuscularly, intravenously, subcutaneously, intrathecally, intraperitoneally, or by direct injection into an organ or a tissue of the subject.

41. The method of any one of claims 36-40, wherein the globin is a gamma-globin.

42. The method of any one of claims 36-41, wherein globin expression in the subject is increased about 9-20%, relative to globin expression in the subject prior to administration of the recombinant nucleic acid or the vector.

43. A host cell comprising the vector according to any one of claim 6-19 or 25-35.

44. A vector comprising a polynucleotide having at least 90%, at least 95%, at least 98%, or at least 99% sequence identity to the nucleic acid sequence of SEQ ID NO: 19.

45. The vector of claim 44, wherein the vector comprises a polynucleotide having a nucleic acid sequence as shown in SEQ ID NO: 19.

46. A vector comprising a polynucleotide having at least 90%, at least 95%, at least 98%, or at least 99% sequence identity to the nucleic acid sequence of SEQ ID NO: 38.

47. The vector of claim 46, wherein the vector comprises a polynucleotide having a nucleic acid sequence as shown in SEQ ID NO: 38.
